(19) United States
(12) Patent Application Publication
(10) Pub. No.: US 2011/0028518 A1
(43) Pub. Date: Feb. 3, 2011

(54) DEXLANSOPRAZOLE PROCESS AND POLYMORPHS

(75) Inventors: Naveen Kumar Kolla, Hyderabad (IN); Nagaraju Manne, Hyderabad (IN); Srinivas Gangula, Hyderabad (IN); Udaykumar Neelam, Hyderabad (IN); Anitha Naredla, Secunderabad (IN); Sudhakar Reddy Baddam, Hyderabad (IN); Sumeet Vishwasrao Patil, Hyderabad (IN); Arjunkumar Tummala, Guntur (IN); Subbareddy Peddireddy, Cuddapah (IN); Sachin Gulabrao Shinde, Hyderabad (IN); Ashok Sigala, Hyderabad (IN); Satish Varma Mudunuru, Hyderabad (IN); Madhu Kiran Tummidi, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, Andhra Pradesh (IN); DR. REDDY'S LABORATORIES, INC., Bridgewater, NJ (US)

(21) Appl. No.: 12/921,873

(22) PCT Filed: Mar. 18, 2009
(86) PCT No.: PCT/US09/37516

§ 371 Date: Sep. 10, 2010

Related U.S. Application Data
(60) Provisional application No. 61/061,882, filed on Jun. 16, 2008.
(60) Provisional application No. 61/096,465, filed on Sep. 12, 2008.
(60) Provisional application No. 61/121,222, filed on Dec. 10, 2008.
(60) Provisional application No. 61/122,109, filed on Dec. 12, 2008.
(60) Provisional application No. 61/154,151, filed on Feb. 20, 2009.

(30) Foreign Application Priority Data
Mar. 18, 2008 (IN) 674/CHE/2008
Jul. 2, 2008 (IN) 1611/CHE/2008
Aug. 19, 2008 (IN) 2016/CHE/2008
Sep. 18, 2008 (IN) 2291/CHE/2008
Dec. 3, 2008 (IN) 3040/CHE/2008

(51) Int. Cl. A61K 31/4439; C07D 401/12; A61P 1/04

(52) U.S. Cl. 514/338; 546/273.7

(57) ABSTRACT

Processes for the preparation of dexlansoprazole, an amorphous form of dexlansoprazole, a solid dispersion of amorphous dexlansoprazole and a pharmaceutically acceptable carrier, and processes for their preparation. Also provided are crystalline compounds 2-[(R)-[(4-chloro-3-methyl-2-pyridinyl)methyl]sulfinyl]-1H-benzimidazole and 2-[(R)-[(4-nitro-3-methyl-2-pyridinyl)methyl]sulfinyl]-1H-benzimidazole, and methods for their preparation.

### INTRODUCTION

The present application relates to processes for the preparation of dexlansoprazole, to amorphous dexlansoprazole, and to processes for preparing amorphous dexlansoprazole. The present application also relates to crystalline 2-[(R)-[(4-chloro-3-methyl-2-pyridinyl)methyl]sulfinyl]-1H-benzimidazole (hereinafter referred to as the “4-chloro analog” of dexlansoprazole) and 2-[(R)-[(4-nitro-3-methyl-2-pyridinyl)methyl]sulfinyl]-1H-benzimidazole (hereinafter referred to as the “4-nitro analog” of dexlansoprazole) and methods for their preparation. The present application also relates to processes for the preparation of crystalline dexlansoprazole.

(R)-(+)-lansoprazole (having the officially adopted name “dexlansoprazole”) is known by its chemical names (R)-2-[[[3-methyl-4-(2,2,2-trifluoroethoxy)-2-pyridinyl]methyl]sulfinyl]-1H-benzimidazole, or (+)-(2)-[(R)-{[3-methyl-4-(2,2,2-trifluoroethoxy)pyridin-2-yl]methyl}sulfinyl]-1H-benzimidazole, and can be represented by structural formula (IA).

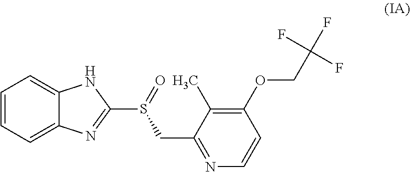

Dexlansoprazole is available in the United States, in products sold by Takeda Pharmaceuticals America, Inc. using the trademark KAPIDEX, for the treatment of symptomatic non-erosive gastroesophageal reflux disease-heartburn associated with gastroesophageal reflux disease (GERD) and erosive esophagitis.

Dexlansoprazole was disclosed in Biochemical Pharmacology (1991), 42(10), 1875-8 and is said to have antisecretory activity due to the inhibition of (H+—K+)-ATPase.

U.S. Pat. No. 5,948,789 discloses a process for enantioselective synthesis of 2-(2-pyridinylmethylsulphinyl)-1H-benzimidazoles or an alkaline salt thereof, in the form of a single enantiomer or in an enantiomerically enriched form, by oxidizing a pro-chiral sulfide with an oxidizing agent in the presence of a chiral titanium complex and a base in an organic solvent.

U.S. Patent Application Publication No. 2005/0288334 A1 discloses a process for preparing an optically pure proton pump inhibitor (PPI) having a sulfinyl structure selected from the group consisting of (S)- or (R)-enantiomers of 5-methoxy-2-[(4-methoxy-3,5-dimethyl-2-pyridinyl)methylsulphinyl]-1H-benzimidazole, 2-[3-methyl-4-(2,2,2-trifluoroethoxy)-2-pyridinyl)methylsulphinyl]-1H-benzimidazole, 2-{[4-[3-methoxypropoxy)-3-methylpyridin-2-yl]methylsulphinyl]-1H-benzimidazole, 5-methoxy-2-((4-methoxy-3,5-dimethyl-2-pyridylmethyl)sulphinyl}-1H-imidazo[4,5-b]pyridine, in enantiomerically pure or enantiomerically enriched form, by oxidizing the corresponding sulfide of said PPI in the presence of a chiral zirconium or a chiral hafnium complex.

International Application Publication No. WO 2005/054228 A1 discloses a process for 2-(2-pyridinylmethylsulphinyl)-1H-benzimidazoles, either as a single enantiomer or in an enantiomerically enriched form by asymmetric oxidation of the corresponding prochiral 4-chloro or 4-nitro analog of 2-(2-pyridinylmethyl-sulphanyl)-1H-benzimidazole with an oxidizing agent and a chiral titanium complex in an organic solvent, followed by reaction of 4-chloro or 4-nitro analog of 2-(2-pyridinylmethylsulphanyl)-1H-benzimidazole with corresponding alkali metal or alkaline earth metal alkoxide. The only example provided in this document describes asymmetric oxidation of 5-methoxy-2-[[(3,5-dimethyl-4-nitro-2-pyridinyl)methyl]thio]-1H-benzimidazole (4-nitro analog of omeprazole) to provide (S)-5-methoxy-2-[[(3,5-dimethyl-4-nitro-2-pyridinyl)methyl]sulfinyl]-1H-benzimidazole and its subsequent reaction with sodium methoxide to get esomeprazole.

U.S. Pat. No. 6,462,058 discloses a crystal of (R)-2-[[[3-methyl-4-(2,2,2-trifluoroethoxy)-2-pyridinyl]methyl]sulfinyl]-1H-benzimidazole (dexlansoprazole), characterized by its X-ray powder diffraction pattern giving interplanar spacings (d) of 11.68, 6.77, 5.84, 5.73, 4.43, 4.09, 3.94, 3.89, 3.69, 3.41, and 3.11 Ångstroms. The patent also discloses a crystal of dexlansoprazole 1.5-hydrate characterized by an X-ray powder diffraction pattern with interplanar spacings (d) of 13.22, 9.60, 8.87, 8.05, 6.61, 5.92, 5.65, 4.49, 3.50 and 3.00 Ångstroms, and is described as more stable and preferable for use as a pharmaceutical than the amorphous form. The patent also discloses processes for the preparation of crystalline dexlansoprazole including, for example, crystallization from solution, crystallization from vapor and crystallization from molten form.

U.S. Patent Application Publication No. 2006/0057195 A1 discloses stable solid dosage form comprising a non-toxic base and an amorphous dexlansoprazole. According to the application, amorphous dexlansoprazole stored with a base has a more stable colour when compared to amorphous dexlansoprazole alone.

The prior methods for the preparation of crystalline dexlansoprazole involves repetitive crystallization operations which require huge quantities of solvent, reducing the commercial viability and ultimately leading to losses in yield and, in turn, making the process uneconomical and not environmentally friendly.

There remains a need to provide improved processes for the preparation of dexlansoprazole, crystalline dexlansoprazole with high yield and high purity directly from the reaction mixture, which is simple, cost-effective, environmentally friendly and commercially viable. There remains a need to a provide stable amorphous form of dexlansoprazole, which may be stored alone without any base as a stabilizer and suitable for a variety of formulations for pharmaceutical use, and a process for its preparation.

### SUMMARY

In an embodiment, the present application provides a process for the preparation of a compound of formula (I),

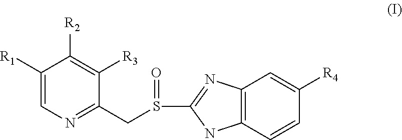

wherein each of R₁, R₂, R₃ and R₄ independently is hydrogen, C₁₋₆ alkyl or C₁₋₆ alkoxy, optionally substituted with one or more fluorine atoms, or C₁₋₆-alkoxy-C₁₋₆ alkoxy groups, or a pharmaceutically acceptable salt thereof, in the form of a single enantiomer or in an enantiomerically enriched form, which includes one or more of the following steps:

a) reacting a compound of formula (II),

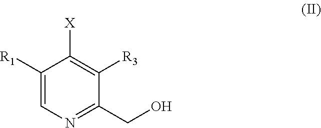

wherein R₁ and R₃ are as described previously, and X is a nitro or halo group; with:

(i) a halogenating agent such as a thionyl halide or phosphorous trihalide; or

(ii) a compound of formula (III),

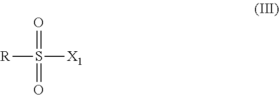

wherein X₁ is a halo group or —OSO₂R, wherein R is an alkyl group, a halogenated alkyl group, or an aryl group, optionally substituted with an alkyl group, to provide a compound of formula (IV) or a salt thereof,

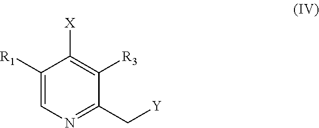

wherein Y is a halo group or —OSO₂R, wherein R is as described previously;

b) reacting a compound of formula (IV) with a 2-mercaptobenzimidazole of formula (V),

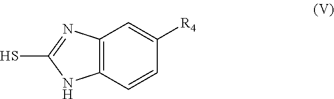

to provide a compound of formula (VI),

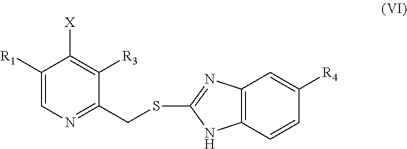

wherein R₁, R₃, R₄ and X are as described previously;

c) enantioselectively oxidizing the compound of formula (VI) with an oxidizing agent in the presence of a chiral auxiliary to provide a compound of formula (VII) in the form of a single enantiomer or in an enantiomerically enriched form,

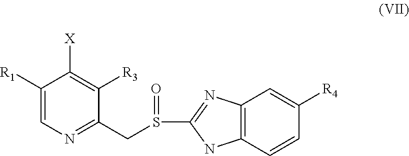

wherein R₁, R₃, R₄ and X are as described previously; and

d) reacting a compound of formula (VII), in the form of a single enantiomer or in an enantiomerically enriched form, with an alkoxide —OZ, wherein Z is a C₁₋₆ alkyl optionally substituted with one or more fluorine atoms, or C₁₋₆-alkoxy-C₁₋₆-alkyl, to provide a compound of formula (I).

In an embodiment, the present application provides a process for the preparation of a substantially pure compound of formula (VII), which includes one or more of the following steps:

a) providing a mixture containing a compound of formula (VII) in a water immiscible solvent;

b) extracting the mixture with an aqueous solution of an organic base;

c) separating the organic phase and adjusting the pH of the aqueous phase with an acid; and

d) isolating a substantially pure compound of formula (VII).

In an embodiment, the present application provides an optical purification of an enantiomerically enriched compound of formula (VII), wherein R₁, R₃, R₄ and X are as described previously, which includes one or more of the following steps:

a) treating an enantiomerically enriched compound of Formula (VII) with a solvent; and

b) isolating the compound of Formula (VII) with an enhanced optical purity.

In an embodiment, the present application provides crystalline 2-[(R)-[(4-chloro-3-methyl-2-pyridinyl)methyl]sulfinyl]-1H-benzimidazole.

In another embodiment, the present application provides crystalline 2-[(R)-[(4-chloro-3-methyl-2-pyridinyl)methyl]sulfinyl]-1H-benzimidazole characterized by a powder X-ray diffraction pattern having peak locations substantially as depicted in Table 1.

In yet another embodiment, the present application provides crystalline 2-[(R)-[(4-chloro-3-methyl-2-pyridinyl)methyl]sulfinyl]-1H-benzimidazole characterized by a powder X-ray diffraction pattern having peak locations substantially as illustrated by FIG. 9, and/or an infrared absorption spectrum having peaks located substantially as illustrated by FIG. 10.

In an embodiment, the present application provides crystalline 2-[(R)-[(4-nitro-3-methyl-2-pyridinyl)methyl]sulfinyl]-1H-benzimidazole.

In another embodiment, the present application provides crystalline 2-[(R)-[(4-nitro-3-methyl-2-pyridinyl)methyl]sulfinyl]-1H-benzimidazole characterized by a powder X-ray diffraction pattern having peak locations substantially as depicted in Table 2.

In another embodiment, the present application provides crystalline 2-[(R)-[(4-nitro-3-methyl-2-pyridinyl)methyl]sulfinyl]-1H-benzimidazole characterized by a powder X-ray diffraction pattern having peak locations substantially as illustrated by FIG. 11, and/or an infrared absorption spectrum having peak locations substantially as illustrated by FIG. 12.

In an embodiment, the present application provides a compound of formula (VII) having a chemical purity of greater than about 95%, or greater than about 97%, or greater than about 98%, as determined using high performance liquid chromatography (HPLC).

In an embodiment, the present application provides a compound of formula (VII) having an enantiomeric purity of greater than about 90%, or greater than about 95%, or greater than about 98%, or greater than about 99%, or greater than about 99.5%, or greater than about 99.8%, or greater than about 99.9%, as determined by HPLC.

In an embodiment, the present application provides an amorphous form of dexlansoprazole.

In another embodiment, the present application provides a process for preparing an amorphous form of dexlansoprazole, which includes one or more of the following steps:

a) providing a solution of dexlansoprazole in a solvent or mixture of solvents; and

b) isolating an amorphous form of dexlansoprazole.

In an embodiment, the present application provides an amorphous form of dexlansoprazole characterized by its X-ray powder diffraction pattern, differential scanning calorimetry (DSC) thermogram, infrared absorption spectrum and/or thermal gravimetric analysis (TGA) curve that respectively may be substantially as illustrated by FIGS. 1, 2, 3 and 4.

In an embodiment, the present application provides an amorphous dexlansoprazole having a water content less than about 5%, or less than about 3%, or less than about 2%, or less than about 1%, or less than about 0.5%, by weight.

In an embodiment, the present application provides amorphous dexlansoprazole, substantially free of residual organic solvents.

In another embodiment, the present application provides a process for the preparation of amorphous dexlansoprazole, substantially free of residual organic solvents, which includes one or more of the following steps:

a) micronizing dexlansoprazole; and

b) drying the product obtained from step a) to provide an amorphous form of dexlansoprazole substantially free of residual organic solvents.

In an embodiment, the present application provides an amorphous form of dexlansoprazole, which is stable during storage.

In an embodiment, the present application provides a process for packaging and storing amorphous dexlansoprazole with increased stability and shelf life, which includes one or more of the following steps:

a) placing dexlansoprazole in a sealed container under an inert atmosphere;

b) placing the sealed container and moisture adsorbent in a second sealed container;

c) placing the second sealed container in a triple laminated bag followed by sealing; and

d) placing the triple laminated bag in a HDPE container and storing in controlled environment chamber at about 2-8° C.

In an embodiment, the present application provides dexlansoprazole having a chemical purity of greater than about 99%, or greater than about 99.4%, or greater than about 99.6%, or greater than about 99.8%, by weight as measured by HPLC.

In an embodiment, the present application provides dexlansoprazole having an enantiomeric purity of greater than about 99%, or greater than about 99.2%, or greater than about 99.4%, or greater than about 99.6%, or greater than about 99.8%, or greater than about 99.9%, by weight as measured by HPLC.

In an embodiment, the present application provides dexlansoprazole having a particle size distribution wherein the 10^{th} volume percentile particle size (D₁₀) is less than about 5 μm, the 50^{th} volume percentile particle size (D₅₀) is less than about 15 μm, or the 90^{th} volume percentile particle size (D₉₀) is less than about 50 μm, or any combination thereof.

In an embodiment, the present application provides dexlansoprazole having a specific surface area more than about 0.5 m²/g, or more than about 1 m²/g, or more than about 2 m²/g, or more than about 3 m²/g, or more than about 5 m²/g.

In an embodiment, the present application provides dexlansoprazole having a bulk density less than about 1 g/ml.

In an embodiment, the present application provides a process for the preparation of crystalline dexlansoprazole, which includes one or more of the following steps:

a) providing a reaction mixture comprising a salt of dexlansoprazole;

b) adjusting the pH of the reaction mixture obtained from step a) with an acid to obtain dexlansoprazole; and

c) isolating crystalline dexlansoprazole from the reaction mixture obtained in step (b).

In an embodiment, the present application provides a solid dispersion of amorphous dexlansoprazole together with one or more pharmaceutically acceptable carriers, with the proviso that the carrier is not a base.

In another embodiment, the present application provides a process for preparing a solid dispersion of amorphous dexlansoprazole together with one or more pharmaceutically acceptable carriers, with the proviso that the carrier is not a base, which includes one or more of the following steps:

a) providing a solution of dexlansoprazole in combination with one or more pharmaceutically acceptable carriers, with the proviso that the carrier is not a base, in a suitable solvent or mixture of solvents;

b) isolating a solid dispersion of amorphous dexlansoprazole together with one or more pharmaceutically acceptable carriers.

An aspect of the present application provides compositions comprising dexlansoprazole substantially free of one or more of its corresponding impurities as measured by HPLC.

An aspect of the present application provides pharmaceutical compositions comprising dexlansoprazole, having particle size distributions wherein the 10^{th} volume percentile particle size (D₁₀) is less than about 5 μm, the 50^{th} volume percentile particle size (D₅₀) is less than about 15 μm, or the 90^{th} volume percentile particle size (D₉₀) is less than about 50 μm, or any combination thereof, together with one or more pharmaceutically acceptable excipients.

An aspect of the present application provides pharmaceutical compositions comprising dexlansoprazole, having specific surface areas more than about 0.5 m²/g, or more than about 1 m²/g, or more than about 2 m²/g, or more than about 3 m²/g, or more than about 5 m²/g, together with one or more pharmaceutically acceptable excipients.

An aspect of the present application provides pharmaceutical compositions comprising dexlansoprazole, having bulk densities less than about 1 g/ml, together with one or more pharmaceutically acceptable excipients.

An aspect of the present application provides pharmaceutical compositions comprising a stable amorphous form of dexlansoprazole together with one or more pharmaceutically acceptable excipients.

Another aspect of the present application provides pharmaceutical compositions comprising a stabilized amorphous solid dispersion of dexlansoprazole together with a pharmaceutically acceptable carrier, with the proviso that the carrier is not a base, optionally with one or more pharmaceutically acceptable excipients.

### BRIEF DESCRIPTION OF THE DRAWINGS

FIG. 1 is an illustration of a powder X-ray diffraction (PXRD) pattern of an amorphous form of dexlansoprazole, prepared according to Example 16 (B).

FIG. 2 is an illustration of an infrared absorption spectrum of an amorphous form of dexlansoprazole, prepared according to Example 16 (B).

FIG. 3 is an illustration of a differential scanning calorimetry (DSC) thermogram of an amorphous form of dexlansoprazole, prepared according to Example 16 (B).

FIG. 4 is an illustration of a thermogravimetric analysis (TGA) curve of an amorphous form of dexlansoprazole, prepared according to Example 16 (B).

FIG. 5 is an illustration of a PXRD pattern of an amorphous solid dispersion of dexlansoprazole with povidone, prepared according to Example 12.

FIG. 6 is an illustration of a PXRD pattern of an amorphous solid dispersion of dexlansoprazole with hydroxypropyl methylcellulose, prepared according to Example 13.

FIG. 7 is an illustration of a PXRD pattern of an amorphous solid dispersion of dexlansoprazole with hydroxypropyl cellulose, prepared according to Example 14.

FIG. 8 is an illustration of a PXRD pattern of an amorphous solid dispersion of dexlansoprazole with croscarmellose sodium, prepared according to Example 15.

FIG. 9 is an illustration of a PXRD pattern of a crystalline 4-chloro analog of dexlansoprazole, prepared according to Example 4.

FIG. 10 is an illustration of an infrared absorption spectrum of a crystalline 4-chloro analog of dexlansoprazole, prepared according to Example 4.

FIG. 11 is an illustration of a PXRD pattern of a crystalline 4-nitro analog of dexlansoprazole, prepared according to Example 1.

FIG. 12 is an illustration of an infrared absorption spectrum of a crystalline 4-nitro analog of dexlansoprazole, prepared according to Example 1.

### DETAILED DESCRIPTION

All percentages and ratios used herein are by weight of the total composition, and all measurements made are at 25° C. and normal pressure unless otherwise designated. All temperatures are in degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Typically, such additives will not be present or will be present only in trace amounts. However, it may be possible to include up to about 10% by weight of materials that could materially alter the basic and novel characteristics of the invention, as long as the utility (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

Note that while the specification may refer to a final product such as, for example, a tablet or other dosage form of the invention as, for example, containing particles having a certain particle size or distribution, or a certain type of, for example, a specific form of a filler, it may be difficult to determine from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

Where this document refers to a material, such as for example, dexlansoprazole, and the unique solid forms, salts, solvates and/or optical isomers thereof by reference to patterns, spectra or other graphical data, it may do so by qualifying that they are “substantially” shown or depicted in a drawing figure, or by one or more data points. By “substantially” used in such a context, it will be appreciated that patterns, spectra and other graphical data can be shifted in their positions, relative intensities, or other values due to a number of factors known to those of skill in the art. For example, in the crystallographic and powder X-ray diffraction arts, some shifts in peak positions or the relative intensities of one or more peaks of a pattern can occur because of, without limitation; the equipment used, the sample preparation protocol, preferred packing and orientations, the radiation source, operator error, method and length of data collection, and the like. However, those of ordinary skill in the art will be able to compare the figures herein with a pattern generated of an unknown form of, for example, dexlansoprazole, and confirm its identity as one of the forms disclosed and claimed herein. The same holds true for other techniques that may be reported herein as well as for distinguishing between amorphous forms.

In addition, where a reference is made to a drawing figure, it is permissible to, and this document includes and contemplates, the selection of any number of data points illustrated in the figure which uniquely define that solid form, salt, solvate, and/or enantiomer within any associated and recited margin of error, for purposes of identification.

A reference to a molecule such as dexlansoprazole, unless otherwise specified or inconsistent with the disclosure in general, refers to any salt, amorphous form, enantiomer and/or solvate form thereof.

When a molecule or other material is identified herein as “pure”, it generally means, unless specified otherwise, that the material is about 99% pure or more. In general, this refers to purity with regard to unwanted residual solvents, reaction byproducts, impurities and unreacted starting materials. In the case of solid forms such as amorphous form, “pure” also means about 99% of one amorphous form free from crystalline forms, as appropriate or in the case of crystalline solids, “pure” also means about 99% of one crystal form free from amorphous forms. “Substantially” pure means, the same as “pure” except that the lower limit is about 98% pure or more and, likewise, “essentially” pure means the same as “pure” except that the lower limit is about 95% pure.

In an embodiment, the present application provides a process for the preparation of a compound of formula (I) or a pharmaceutically acceptable salt thereof, in the form of a single enantiomer or in an enantiomerically enriched form,

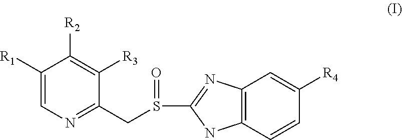

wherein each of R₁, R₂, R₃ and R₄ may independently be hydrogen, C₁₋₆ alkyl, C₁₋₆ alkoxy optionally substituted with one or more fluorine atoms, or a C₁₋₆-alkoxy-C₁₋₆ alkoxy group, which includes one or more of the following steps:

a) reacting a compound of formula (II),

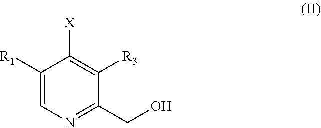

wherein R₁ and R₃ are as described previously, and X is nitro or halo; with

(i) a halogenating agent such as thionyl halide or a phosphorous trihalide; or

(ii) a compound of formula (III),

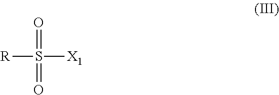

wherein X₁ is halo or —OSO₂R, wherein R may be an alkyl group, a halogenated alkyl group, or an aryl group optionally substituted with an alkyl group, to provide a compound of formula (IV) or its salt;

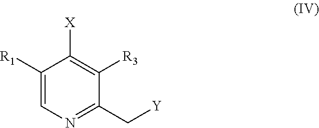

wherein Y is halo or —OSO₂R, and R is as described previously;

b) reacting a compound of formula (IV) with 2-mercaptobenzimidazole of formula (V),

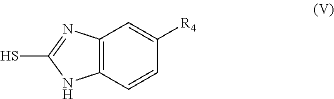

to provide a compound of formula (VI),

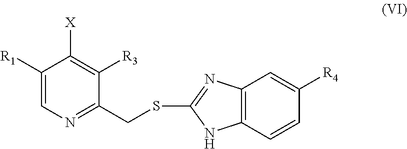

wherein R₁, R₃, R₄ and X are as described previously;

c) enantioselectively oxidizing a compound of formula (VI) with an oxidizing agent in the presence of a chiral auxiliary to provide a compound of formula (VII) in the form of a single enantiomer or in an enantiomerically enriched form,

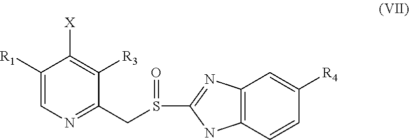

wherein R₁, R₃, R₄ and X are as described previously; and

d) reacting a compound of formula (VII) in the form of a single enantiomer or in an enantiomerically enriched form with an alkoxide —OZ,

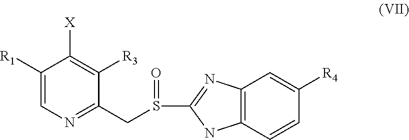

wherein Z may be C₁₋₆ alkyl optionally substituted with one or more fluorine atoms, or C₁₋₆-alkoxy-C₁₋₆-alkyl, to provide a compound of formula (I).

Step a) involves reacting a compound of formula (II) with a halogenating agent such as thionyl halides or phosphorous trihalides or a compound of formula (III) to provide a compound of formula (IV) or its salts.

Step (a) may be carried out by any techniques known in the art.

Step a) may be optionally carried out in a suitable solvent, which is inert to the intended reaction. Suitable solvents that may be used in step a) include but are not limited to: esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step a) may be optionally carried out in the presence of a base. Suitable bases that may be used in step a) include but are not limited to: organic bases such as triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, and cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like; alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, magnesium, substituted or unsubstituted ammonium and the like; and any other suitable bases.

Suitable temperatures that may be used in step a) may be less than about 200° C., or less than about 150° C., or less than about 100° C., or less than about 60° C., or any other suitable temperatures.

After completion of the reaction, the compound of formula (IV) may be isolated from the reaction mixture or the reaction mixture obtained in step (a) containing compound of formula (IV) may be directly used in step b).

In one variant, when step a) is conducted by reacting a compound of formula (II), wherein X is nitro, with a halogenating agent such as thionyl halides or phosphorous trihalides to get the corresponding compound of formula (IV), wherein X is nitro and Y is halo, there may be possibility for the presence of a compound of formula (IV), wherein X is halo and Y is halo, as an impurity in a desired compound of formula (IV), wherein X is nitro and Y is halo. A compound of formula (IV), wherein X is nitro and Y is halo, contaminated with a compound of formula (IV), wherein X is halo and Y is halo, may be purified to bring down the level of compound of formula (IV), wherein X is halo and Y is halo, to a desired level, prior to use for the reaction in step b), or it may be used directly for the reaction in step b) without further purification, both of which are within the scope of the present application.

Step b) involves reacting a compound of formula (IV) with a 2-mercaptobenzimidazole having formula (V) to provide a compound of formula (VI).

Step b) may be carried out by any technique known in the art.

Step b) may be optionally carried out in a suitable solvent. Suitable solvents that may be used in step b) include but are not limited to: water; alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol and the like; ketones such as acetone, butanone; 2-pentanone, 3-pentanone, methyl butyl ketone, methyl iso-butyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step b) may be optionally carried out in presence of a base. Suitable bases that may be used in step b) include but are not limited to: organic bases such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and any other suitable bases.

Suitable temperatures that may be used in step b) may be less than about 200° C., or less than about 150° C., or less than about 100° C., or less than about 60° C., or any other suitable temperatures.

If desired, step b) may be carried out under phase transfer catalyzed conditions, in the presence of a phase transfer catalyst. Such phase transfer catalyzed conditions may include but are not limited to solid-liquid phase transfer catalyzed conditions or liquid-liquid phase transfer catalyzed conditions.

The resulting compound of formula (VI) may have a purity of more than about 95%, or more than about 98%, or more than about 99%, or more than about 99.5%. A compound of formula (VI) may be substantially free one or more of the following compounds as impurities.

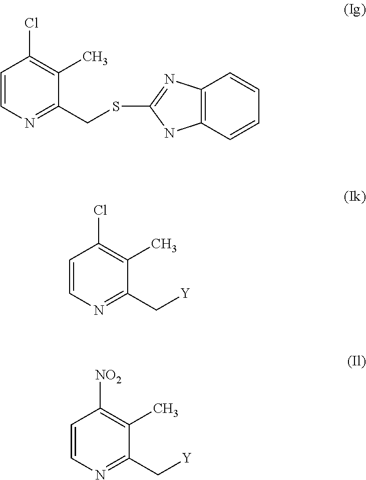

wherein Y is as described previously.

Step c) involves enantioselective oxidation of a compound of formula (VI) with an oxidizing agent in the presence of a chiral auxiliary to provide a compound of formula (VII) in the form of a single enantiomer or in an enantiomerically enriched form.

Suitable oxidizing agents that may be used in step c) include but are not limited to: hydroperoxide reagents such as t-butylhydroperoxide, cumene hydroperoxide, hydrogen peroxide and the like; peracids such as peracetic acid, m-chloroperbenzoic acid, perphthalic acid, ε-phthalimidoperhexanoic acid and the like; sodium perborate and the like; and any other suitable oxidizing agents.

The quantity of oxidizing agent that may be used may range from about 0.1 to about 3 molar equivalents, or any other suitable quantity, per molar equivalent of the compound of formula (VI).

Suitable chiral auxiliaries that may be used in step c) for enantioselective oxidation of a pro-chiral sulfide of formula (VI) include but are not limited to: chiral transition metal complexes such as chiral titanium complexes, chiral zirconium complexes, chiral vanadium complexes, chiral hafnium complexes, and the like, and any other suitable chiral metal complexes.

The chiral transition metal complexes may be prepared from chiral ligands and transition metal compounds.

The chiral auxiliaries that may be used in step c) may be prepared in the presence or absence of a pro-chiral sulfide of formula (VI).

The transition metal compounds used for the preparation of the chiral transition metal complexes include but are not limited to: titanium(IV) isopropoxide, titanium(IV) propoxide, titanium(IV) ethoxide and titanium(IV) methoxide; zirconium(IV) acetylacetonate, zirconium(IV) butoxide, zirconium(IV) t-butoxide, zirconium(IV) ethoxide, zirconium(IV) n-propoxide and zirconium(IV) isopropoxide; vanadium oxytripropoxide, vanadium oxyisopropoxide and vanadyl acetylacetonate; hafnium(IV) acetylacetonate, hafnium(IV) butoxide, hafnium(IV) n-propoxide, hafnium(IV) isopropoxide, hafnium(IV) ethoxide, hafnium(IV) t-butoxide, iron based compounds; and the like, and any other suitable metal compounds.

The amount of transition metal compound that may be used in step c) may range from about 0.1 to about 3 molar equivalents, or any other suitable quantity, per molar equivalent of the compound of Formula (VI).

The chiral ligands that may be used for the preparation of chiral transition metal complexes include but are not limited to: chiral alcohols, such as binaphthol; mandelic acid; hydrobenzoin; esters of tartaric acid such as (+)-dialkyl-L-tartrates or (−)-dialkyl-D-tartrates, including (+)-dimethyl-L-tartrate, (−)-dimethyl-D-tartrate, (+)-diethyl-L-tartrate, (−)-diethyl-D-tartrate, (+)-diisopropyl-L-tartrate, (−)-diisopropyl-D-tartrate, (+)-dibutyl-L-tartrate, (−)-dibutyl-D-tartrate, (+)-di-t-butyl-L-tartrate, (−)-di-t-butyl-D-tartrate; and any other suitable chiral ligands.

The amount of chiral ligand that may be used may range from about 0.1 to about 6 molar equivalents, or any other suitable quantity, per molar equivalent of the compound of formula (VI).

Step c) may be optionally carried out in the presence of water in order to improve the enantioselectivity of the reaction to provide a compound of formula (VII) with greater enantiomeric purity. For this, either water may be used in the preparation of a chiral transition metal complex, which may be in turn used for the reaction in step c), or water may be added to the reaction mixture comprising a chiral transition metal complex and a compound of formula (VI). The amount of water that may be used in step c) may range from about 0.1 to about 1 molar equivalent, per molar equivalent of the compound of Formula (VI).

Step c) may be optionally carried out in a suitable solvent. Suitable solvents that may be used in step c) include but are not limited to: ketones such as acetone, butanone; 2-pentanone, 3-pentanone, methyl butyl ketone, methyl iso-butyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step c) may be optionally carried out in the presence of a base. Suitable bases that may be used in step c) include but are not limited to: organic bases such as triethylamine, tributylamine, N,N-diisopropylethylamine, N-methylpyrrolidine, pyridine, 4-(N,N-dimethylamino)pyridine, N-methylmorpholine, morpholine, imidazole, 2-methyl imidazole, 4-methylimidazole and the like; inorganic bases such as alkali metal hydroxides such as lithium hydroxide, sodium hydroxide, potassium hydroxide, and cesium hydroxide; alkaline hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; and ion exchange resins including resins bound to ions such as sodium, potassium, lithium, calcium, magnesium, substituted or unsubstituted ammonium and the like; and any other suitable bases.

The quantities of base that may be used for enantioselective oxidation of a pro-chiral sulfide of formula (VI) in step c) may range from about 0.02 to about 3 molar equivalents, or any other suitable quantity, per molar equivalent of the compound of formula (VI).

Enantioselective oxidation of a pro-chiral sulfide of formula (VI) in step c) may be carried out at temperatures less than about 100° C., or less than about 50° C., or less than about 30° C., or less than about 10° C., or less than about 5° C., or less than about 0° C., or less than about −5° C., or less than about −10° C., or less than about −20° C., or any other suitable temperatures. Suitable temperatures that may be used for the preparation of a chiral transition metal complex may be less than about 200° C., or less than about 150° C., or less than about 100° C., or less than about 80° C., or less than about 60° C., or less than about 40° C., or any other suitable temperatures.

The compound of formula (VII) obtained in step c) may be crystalline, or amorphous, or a mixture thereof.

Step d) involves reacting a compound of formula (VII) in the form of a single enantiomer or in an enantiomerically enriched form with an alkoxide —OZ to provide a compound of formula (I).

Step d) may be optionally carried out in a suitable solvent. Suitable solvents that may be used in step d) include but are not limited to: ketones such as acetone, butanone; 2-pentanone, 3-pentanone, methyl butyl ketone, methyl iso-butyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step d) may be optionally carried out in the presence of a base. Suitable bases that may be used in step d) include but are not limited to: organic bases such as triethylamine, tributylamine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide, n-butyl lithium, lithium diisopropylamide and the like; alkali metal hydroxides such as lithium hydroxide, sodium hydroxide, potassium hydroxide, and cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like; alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, magnesium, substituted or unsubstituted ammonium and the like; and any other suitable bases.

Step d) may be carried out at temperatures less than about 250° C., or less than about 200° C., or less than about 150° C., or less than about 120° C., or less than about 100° C., or less than about 80° C., or less than about 60° C., or less than about 40° C., or any other suitable temperatures.

In an embodiment, the present application provides processes for the preparation of a substantially pure compound of formula (VII), which include one or more of the following steps:

a) providing a mixture containing a compound of formula (VII) in a water immiscible solvent;

b) extracting the mixture with an aqueous solution of an organic base;

c) separating the organic phase and adjusting the pH of the aqueous phase with an acid; and

d) isolating the substantially pure compound of formula (VII).

Step a) involves providing a mixture containing a compound of formula (VII) in a water immiscible solvent.

The mixture containing a compound of formula (VII) in a water immiscible solvent in step a) may be obtained directly from a reaction mixture containing the compound of formula (VII), optionally after adding a water immiscible solvent.

Alternatively, the mixture containing a compound of formula (VII) in water immiscible solvent in step a) may be obtained by adding compound of formula (VII) to a water immiscible solvent.

Suitable water immiscible solvents that may be used in step a) include but are not limited to: ketones such as methyl isobutyl ketone and the like; esters such as methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, 1,2-dimethoxyethane, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; and mixtures thereof.

Optionally, the above water immiscible solvent may also contain a water miscible solvent, in an amount that does not affect the intended extraction process in step b).

Step b) involves extraction of the mixture from step a) with an aqueous solution of an organic base.

Suitable organic bases that may be used in step b) include, but are not limited to, ammonia, methylamine, dimethylamine, ethylamine, diethylamine, trimethylamine, triethylamine, t-butyl amine, tributylamine, N,N-diisopropylethylamine, N-methylpyrrolidine, piperidine, pyrrolidine, pyridine, 4-(N,N-dimethylamino)pyridine, N-methylmorpholine, morpholine, imidazole, 2-methylimidazole, 4-methylimidazole, and the like, and any other suitable bases.

Step c) involves separating the organic phase and adjusting the pH of the aqueous phase with an acid.

An acid may be used to adjust the pH in step c). Suitable acids that may be used in step c) include but are not limited to: organic acids such as acetic acid, formic acid, trifluoroacetic acid, chloroacetic acid, propionic acid, butanoic acid, isobutyric acid, valeric acid, isovaleric acid, benzoic acid, salicylic acid, phthalic acid, p-toluene sulphonic acid, o-toluene sulphonic acid, benzene sulphonic acid, methane sulphonic acid, ethane sulphonic acid and the like; ion exchange resins such as resins bound to acids such as p-toluene sulphonic acid, sulphuric acid, phosphoric acid, styrene-divinylbenzenesulfonic acid and the like; chelated resins; neutral resins; and any other reagent which may bring the pH in step c) to the desired level without affecting the quality of compound of formula (VII). Optionally, the aqueous phase may be washed with a water immiscible solvent, such as a solvent described in step a), before pH adjustment. pH may be adjusted to about 7 to about 9, or any other suitable pH, which may dissociate the salt that may be present in the aqueous phase before pH adjustment. Optionally, a water miscible solvent may be added to the aqueous phase before or after pH adjustment. The water miscible solvents that may be added include but are not limited to: alcohols such as methanol, ethanol, 1-propanol, and the like; ketones such as acetone, and the like; ethers such as tetrahydrofuran, 1,4-dioxane, and the like; nitriles such as acetonitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step d) involves isolating the substantially pure compound of formula (VII).

Isolation of a substantially pure compound of formula (VII) in step d) may involve methods including removal of solvent, cooling, concentrating the reaction mass, adding an anti-solvent, extraction with a solvent and the like. Stirring or other alternate methods such as shaking, agitation and the like, may also be employed for isolation.

The isolated compound of formula (VII) may be recovered by methods including decantation, centrifugation, gravity filtration, suction filtration or any other techniques for the recovery of solids. The compound of formula (VII) thus isolated may carry some amount of occluded mother liquor and may have higher than desired levels of impurities. If desired, it may be washed with a solvent or a mixture of solvents to wash out the impurities.

The recovered solid may be optionally further dried. Drying may be carried out in a tray dryer, vacuum oven, air oven, fluidized bed dryer, spin flash dryer, flash dryer and the like. The drying may be carried out at temperatures less than about 150° C., or less than about 120° C., or less than about 100° C., or less than about 80° C., or less than about 60° C., or any other suitable temperatures as long as the compound of formula (VII) is not degraded in quality; at atmospheric pressure or under a reduced pressure. The drying may be carried out for any desired times until the required purity is achieved. For example, it may vary from about 1 to about 8 hours, or longer.

In an embodiment, the present application provides a compound of formula (VII) having a chemical purity of greater than about 95%, or greater than about 97%, or greater than about 98%, by weight as measured by HPLC.

In an embodiment, the present application provides a compound of formula (VII) having an enantiomeric purity of greater than about 90%, or greater than about 95%, or greater than about 98%, or greater than about 99%, or greater than about 99.5%, or greater than about 99.8%, or greater than about 99.9%, by weight as measured by HPLC.

“Substantially pure compound of Formula (VII)” as used herein, unless otherwise defined refers to the compound containing less than about 5%, or less than about 3%, or less than about 2%, or less than about 1%, or less than about 0.5%, or less than about 0.2%, or less than about 0.1%, by weight of one or more of its corresponding impurities and containing a total amount of impurities of less than about 2%, or less than about 1%, or less than about 0.5%, or less than about 0.3%, or less than about 0.1%, or less than about 0.05%, by weight as measured by HPLC. Impurities, as used herein, unless otherwise defined refer to the compounds of formula VIIIa and formula VIIb, unwanted enantiomers, or any other possible residual impurity.

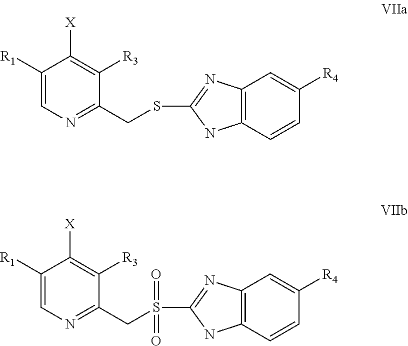

In one variant, 2-[(R)-[(4-nitro-3-methyl-2-pyridinyl)methyl]sulfinyl]-1 h-benzimidazole of the present application may contain one or both of the following impurities (Ib) and (Ic).

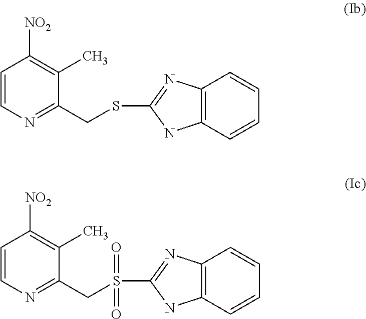

In another variant, 2-[(R)-[(4-chloro-3-methyl-2-pyridinyl)methyl]sulfinyl]-1H-benzimidazole of the present application may contain one or both of the following impurities (Ig) and (Ih).

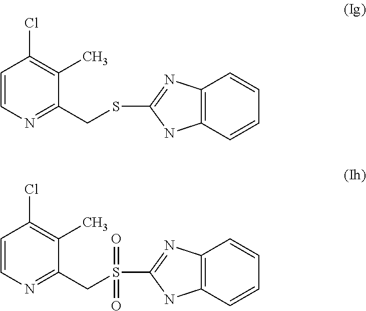

In an embodiment, the present application provides processes for optical purification of an enantiomerically enriched compound of formula (VII), which includes one or more of the following steps:

a) treating an enantiomerically-enriched compound of formula (VII) with a suitable solvent; and

b) isolating the compound of formula (VII) with an enhanced optical purity.

Step a) involves treating an enantiomerically enriched compound of formula (VII) with a suitable solvent.

Suitable solvents that may be used in step a) include but are not limited to: water; alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol and the like; ketones such as acetone, butanone; 2-pentanone, 3-pentanone, methyl butyl ketone, methyl isobutyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; acids such as formic acid, acetic acid, propionic acid, valeric acid and the like; and mixtures thereof.

The reaction mixture obtained in step a) may be optionally filtered to remove any insoluble solids, or particles may be removed by other methods such as decantation, centrifugation, gravity filtration, suction filtration or any other technique for the removal of solids.

Optionally, step a) may be accompanied by precipitation of the racemic compound of formula (VII). The precipitated racemate may be removed by methods such as decantation, centrifugation, gravity filtration, suction filtration or any other technique for the removal of solids.

Suitable temperatures that may be used in step a) may be less than about 150° C., or less than about 100° C., or less than about 80° C., or less than about 60° C., or less than about 40° C., or less than about 20° C., or less than about 0° C., or less than about −20° C., or any other suitable temperatures.

Step b) involves isolating the compound of formula (VII) with an enhanced optical purity.

The isolation in step b) may be effected by methods including removal of solvent, cooling, concentrating the reaction mass, adding an anti-solvent and the like. The suitable temperatures for isolation may be less than about 100° C., or less than about 60° C., or less than about 40° C., or less than about 20° C., or less than about 5° C., or less than about 0° C., or less than about −10° C., or less than about −20° C., or any other suitable temperatures. Suitable times for isolation may be less than about 5 hours, or less than about 3 hours, or less than about 2 hours, or less than about 1 hour, or longer times may be used. However, the exact temperatures and times required for complete isolation may be readily determined by a person skilled in the art and will also depend on parameters such as concentration and temperature of the solution or slurry. Stirring or other alternate methods such as shaking, agitation and the like, may also be employed for isolation.

Suitable techniques that may be used for the removal of solvent include but are not limited to rotational distillation using a device such as Buchi Rotavapor, spray drying, agitated thin film drying (“ATFD”), freeze drying (lyophilization) and the like, optionally under reduced pressure.

The isolated compound of formula (VII) may be recovered by methods including decantation, centrifugation, gravity filtration, suction filtration or any other technique for the recovery of solids. The compound of formula (VII) thus isolated may carry some amount of occluded mother liquor and thus have higher than desired levels of impurities. If desired, the solid may be washed with a suitable solvent or a mixture of solvents such as those used in step a) to wash out the impurities.

The recovered solid may be optionally further dried. Drying may be carried out in a tray dryer, vacuum oven, air oven, fluidized bed dryer, spin flash dryer, flash dryer and the like. The drying may be carried out at temperatures less than about 150° C., or less than about 120° C., or less than about 100° C., or less than about 80° C., or less than about 60° C., or any other suitable temperatures as long as the compound of formula (VII) is not degraded in quality, at atmospheric pressure or under a reduced pressure. The drying may be carried out for any desired time until the required purity is achieved. For example, it may vary from about 1 to about 8 hours, or longer.

In an embodiment, the present application provides crystalline 2-[(R)-[(4-chloro-3-methyl-2-pyridinyl)methyl]sulfinyl]-1H-benzimidazole.

In another embodiment, the present application provides crystalline 2-[(R)-[(4-chloro-3-methyl-2-pyridinyl)methyl]sulfinyl]-1H-benzimidazole characterized by its powder X-ray diffraction peaks located substantially as depicted in Table 1.

TABLE 1

| 2θ (degrees) ± 0.2 | d-spacing (Å) ± 0.02 |
|---|---|
| 6.6 | 13.21 |
| 11.6 | 7.61 |
| 12.2 | 7.23 |
| 13.4 | 6.55 |
| 14.5 | 6.07 |
| 15.2 | 5.81 |
| 17.2 | 5.14 |
| 18.5 | 4.79 |
| 19.5 | 4.53 |
| 20.2 | 4.37 |
| 22.1 | 4.01 |
| 22.7 | 3.91 |
| 23.6 | 3.75 |
| 23.9 | 3.70 |
| 24.8 | 3.57 |
| 26.2 | 3.39 |
| 26.8 | 3.32 |
| 27.1 | 3.28 |
| 28.4 | 3.13 |
| 29.4 | 3.02 |
| 30.4 | 2.93 |
| 31.1 | 2.86 |
| 33.7 | 2.65 |
| 36.0 | 2.48 |
| 37.5 | 2.39 |
| 39.0 | 2.30 |
| 40.2 | 2.24 |
| 41.9 | 2.15 |

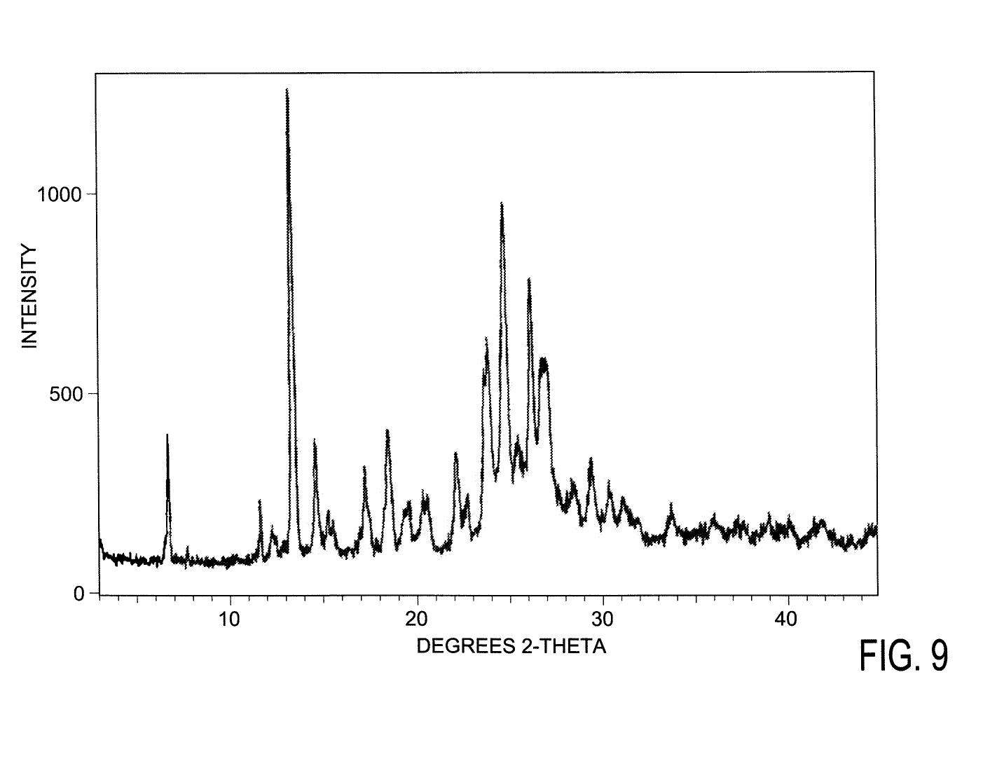

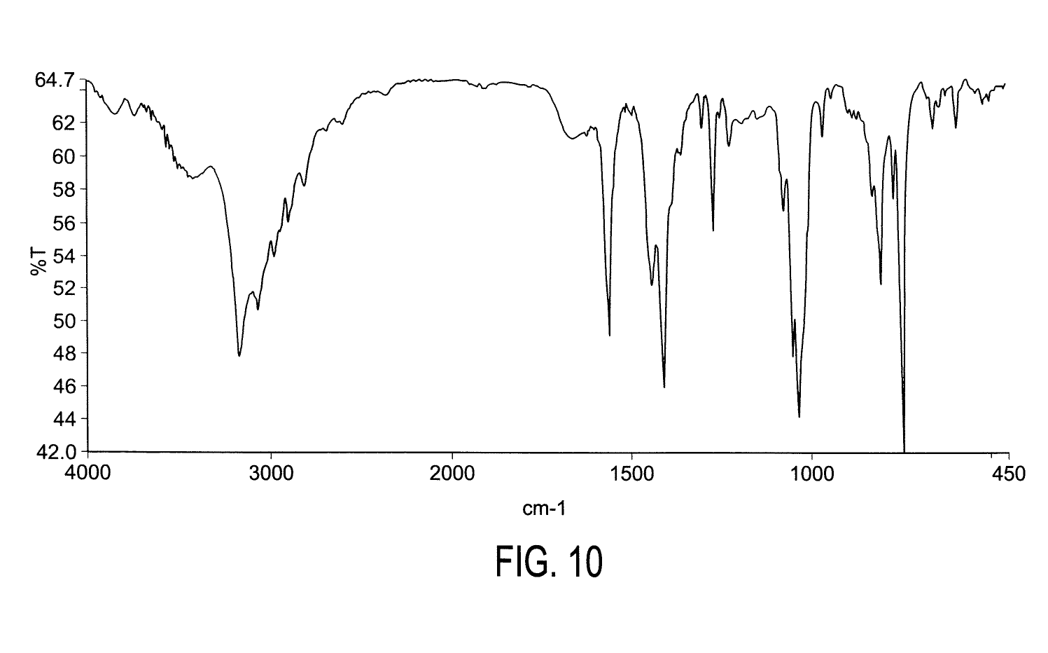

In yet another embodiment, the present application provides crystalline 2-[(R)-[(4-chloro-3-methyl-2-pyridinyl)methyl]sulfinyl]-1H-benzimidazole, characterized by its powder X-ray diffraction pattern having peaks located substantially as illustrated by FIG. 9, and/or an infrared absorption spectrum having peaks located substantially as illustrated by FIG. 10.

In an embodiment, the present application provides crystalline 2-[(R)-[(4-nitro-3-methyl-2-pyridinyl)methyl]sulfinyl]-1H-benzimidazole.

In another embodiment, the present application provides crystalline 2-[(R)-[(4-nitro-3-methyl-2-pyridinyl)methyl]sulfinyl]-1H-benzimidazole, characterized by its powder X-ray diffraction with peaks located substantially as depicted in Table 2.

TABLE 2

| 2θ (degrees) ± 0.2 | d-spacing (Å) ± 0.02 |
|---|---|
| 3.2 | 26.77 |
| 6.5 | 13.47 |
| 9.8 | 8.99 |
| 16.0 | 5.50 |
| 16.5 | 5.33 |
| 17.0 | 5.20 |
| 17.7 | 4.98 |
| 18.3 | 4.82 |
| 19.3 | 4.58 |
| 19.6 | 4.50 |
| 20.0 | 4.41 |
| 23.8 | 3.72 |
| 23.9 | 3.71 |
| 24.1 | 3.67 |
| 24.4 | 3.63 |
| 24.9 | 3.56 |
| 25.4 | 3.49 |
| 26.1 | 3.40 |
| 26.8 | 3.32 |
| 28.5 | 3.12 |
| 29.0 | 3.06 |
| 29.6 | 3.01 |
| 30.4 | 2.93 |
| 31.8 | 2.81 |
| 32.6 | 2.73 |
| 33.1 | 2.70 |
| 33.5 | 2.67 |
| 34.0 | 2.63 |
| 35.0 | 2.55 |
| 36.0 | 2.48 |
| 36.5 | 2.45 |
| 37.0 | 2.42 |
| 37.6 | 2.38 |
| 38.0 | 2.36 |
| 39.0 | 2.30 |
| 39.9 | 2.25 |
| 40.7 | 2.21 |
| 41.4 | 2.17 |
| 42.3 | 2.13 |
| 43.5 | 2.07 |

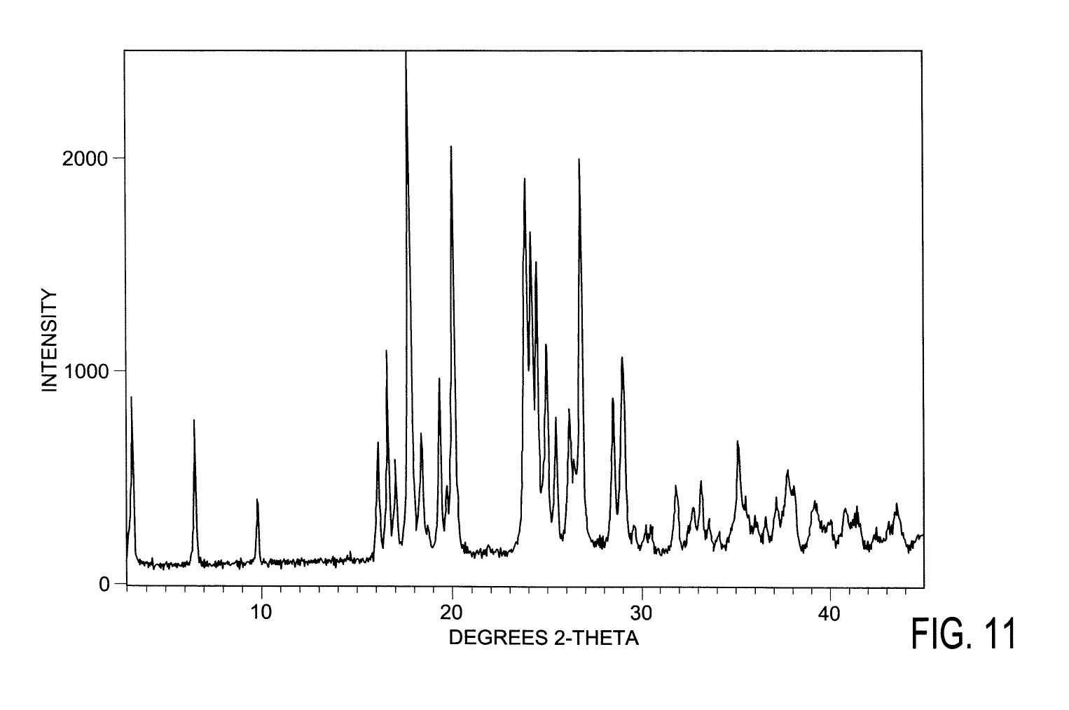

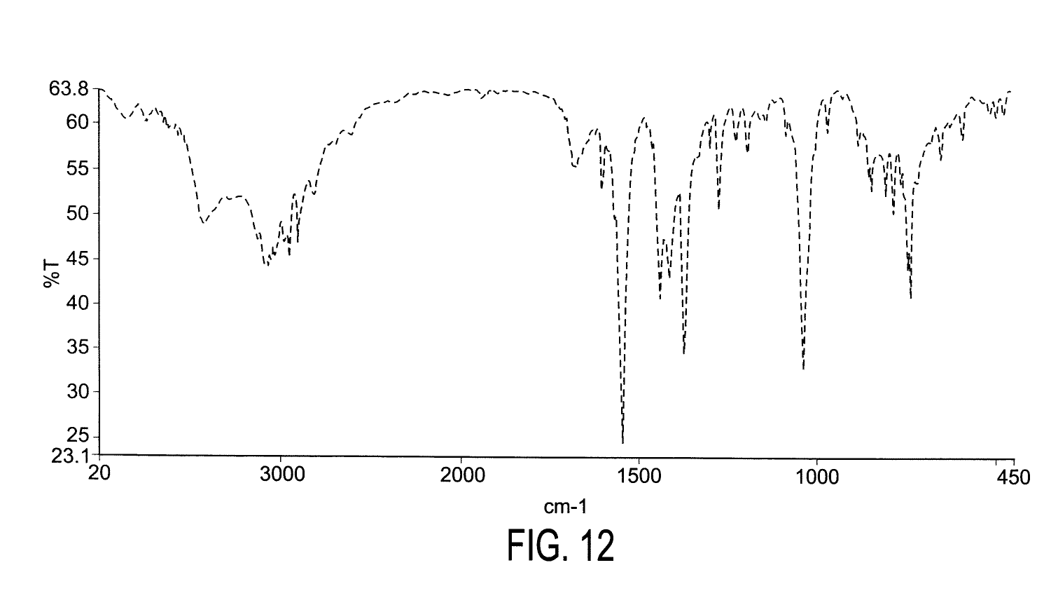

In yet another embodiment, the present application provides crystalline 2-[(R)-[(4-nitro-3-methyl-2-pyridinyl)methyl]sulfinyl]-1H-benzimidazole, characterized by its powder X-ray diffraction pattern having peak locations substantially as illustrated by FIG. 11, and/or infrared absorption spectrum peaks located substantially as illustrated by FIG. 12.

A crystalline 4-chloro analog of dexlansoprazole, or crystalline 4-nitro analog of dexlansoprazole, of the present application is useful as an intermediate in processes for preparation of pure dexlansoprazole with a desired quality.

In an embodiment, the present application provides an amorphous form of dexlansoprazole.

In another embodiment, the present application provides a process for preparing an amorphous form of dexlansoprazole, which includes one or more of the following steps:

a) providing a solution of dexlansoprazole in a solvent or mixture of solvents;

b) isolating the amorphous form of dexlansoprazole.

Step a) involves providing a solution of dexlansoprazole in a solvent or mixture of solvents.

Providing a solution in step a) includes:

i) direct use of a reaction mixture containing dexlansoprazole that is obtained in the course of its synthesis; or

ii) dissolving dexlansoprazole in a suitable solvent or mixture of solvents.

Any physical form of dexlansoprazole, such as crystalline, amorphous or their mixtures may be utilized for providing the solution of dexlansoprazole in step a).

Suitable solvents that may be used in step a) include but are not limited to: water; alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol and the like; ketones such as acetone, butanone; 2-pentanone, 3-pentanone, methyl butyl ketone, methyl isobutyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; chlorinated hydrocarbons such as dichloromethane, chloroform, 1,1,2-trichloroethane, 1,2-dichloroethene and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

The dissolution temperatures may range from about −20° C. to about the reflux temperature of the solvent, depending on the solvent used for dissolution, as long as a clear solution of dexlansoprazole is obtained without affecting its quality.

The solution may optionally be treated with carbon, flux-calcined diatomaceous earth (Hyflow) or any other suitable material to remove colour and/or to get clarity of the solution.

Optionally, the solution obtained above may be filtered to remove any insoluble particles. The insoluble particles may be removed suitably by filtration, centrifugation, decantation or any other suitable techniques. The solution may be filtered by passing through paper, glass fiber, or other membrane material, or a bed of a clarifying agent such as celite or Hyflow. Depending upon the equipment used and the concentration and temperature of the solution, the filtration apparatus may need to be preheated to avoid premature crystallization.

Step b) involves isolation of an amorphous form of dexlansoprazole from the solution of step a).

In one variant, the isolation may be affected by removing solvent. Suitable techniques which may be used for the removal of solvent include using a rotational distillation device such as a Buchi Rotavapor, spray drying, agitated thin film drying (“ATFD”), freeze drying (lyophilization), and the like or any other suitable technique.

The solvent may be removed, optionally under reduced pressures, at temperatures less than about 200° C., or less than about 150° C., or less than about 100° C., or less than about 60° C., or less than about 40° C., or less than about 20° C., or less than about 0° C., or less than about −20° C., or less than about −40° C., or less than about −60° C., or less than about −80° C., or any other suitable temperatures.

Freeze drying (lyophilization) may be carried out by freezing a solution of dexlansoprazole at low temperatures required to freeze the solution of dexlansoprazole and reducing the pressure as required to remove the solvent from the frozen solution of dexlansoprazole. Temperatures that may be required freeze the solution, depending on the solvent chosen to make the solution of dexlansoprazole, may range from about −80° C. to about 0° C., or up to about 40° C. Temperatures that may be required to remove the solvent from the frozen solution may be less than about 20° C., or less than about 0° C., or less than about −20° C., or less than about −40° C., or less than about −60° C., or less than about −80° C., or any other suitable temperatures.

Alternatively, isolation may also be effected by adding a suitable anti-solvent to the solution obtained in step a), optionally after concentrating the solution obtained in step a). Suitable anti-solvents that may be used include but are not limited to: ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; aromatic hydrocarbons such as toluene, xylenes, chlorobenzene, tetraline and the like; and mixtures thereof.

The compound obtained from step b) may be collected using techniques such as by scraping, or by shaking the container, or other techniques specific to the equipment used.

The product thus isolated may be optionally further dried to afford the amorphous form of dexlansoprazole.

Drying may be suitably carried out in a tray dryer, vacuum oven, Buchi Rotavapor, air oven, fluidized bed dryer, spin flash dryer, flash dryer and the like. The drying may be carried out at temperatures of less than about 200° C., or less than about 150° C., or less than about 100° C., or less than about 60° C., or less than about 40° C., or less than about 20° C., or less than about 0° C., or less than about −20° C., or any other suitable temperatures, at atmospheric pressure or under reduced pressures. The drying may be carried out for any time period required for obtaining a desired quality, such as from about 15 minutes to several hours.

The dried product may be optionally milled to get desired particle sizes. Milling or micronization may be performed before drying, or after the completion of drying of the product. Techniques that may be used for particle size reduction include, without limitation, ball, roller and hammer mills, and jet mills.

In one variant, the temperature that may be used in various operations in the preparation of stable amorphous form of dexlansoprazole of the present application plays a major role in the formation of impurity at RRT (relative retention time) of 1.98, as characterized by HPLC, to an undesired level for a pharmaceutical product. The said impurity at RRT 1.98 by HPLC has a mass number of m/z 467 as characterized by LC-MS (liquid chromatography-mass spectrum) analysis.

The structure of the impurity at RRT 1.98 is estimated to be one of (A) or (B).

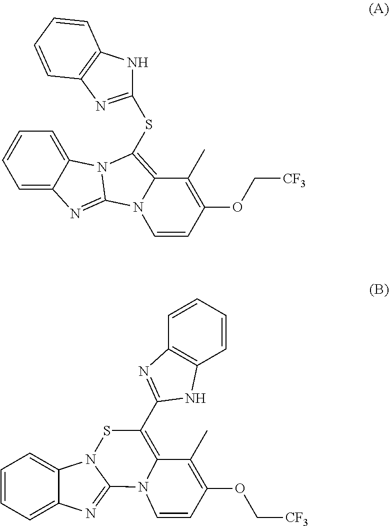

The temperatures that may be suitably used for various operations in the preparation of an amorphous form of dexlansoprazole of the present application may be less than about 60° C., or less than about 50° C., or less than about 40° C., in order to control the impurity at RRT 1.98, as characterized by HPLC, to a concentration of less than about 0.2% by weight as measured by HPLC.

The “various operations” as used herein include but are not limited to dissolution, stirring, distillation, evaporation, filtration, drying, milling and storing.

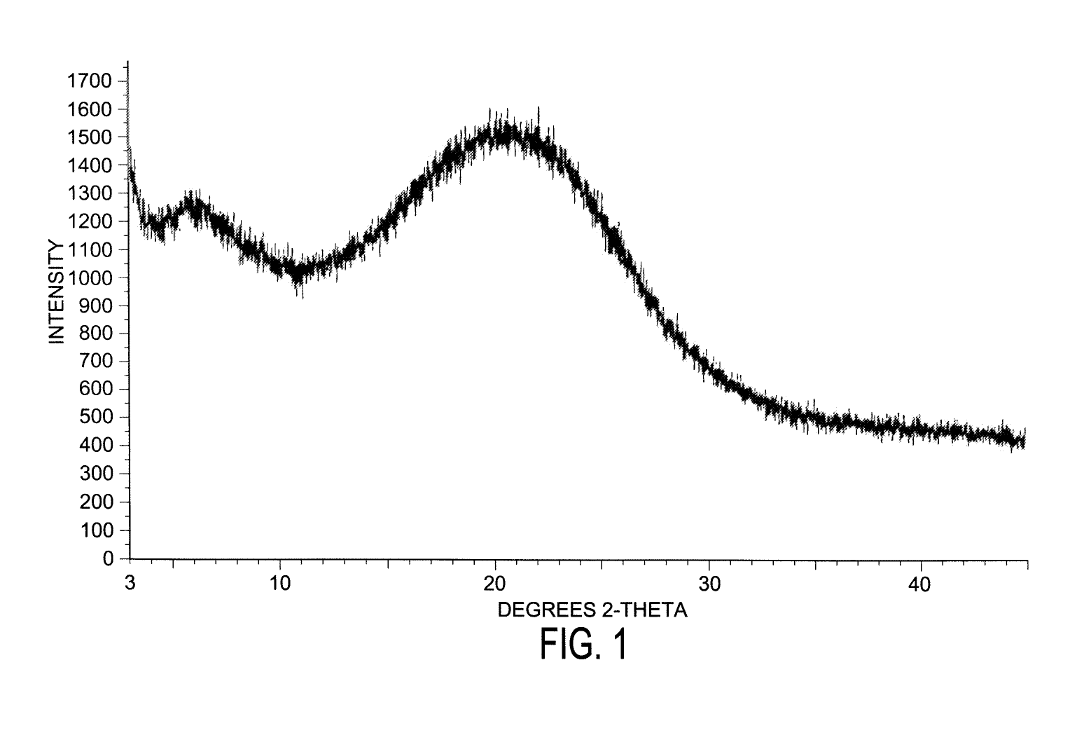

In an embodiment, the present application provides an amorphous form of dexlansoprazole characterized by its X-ray powder diffraction pattern, infrared absorption spectrum, differential scanning calorimetry (DSC) thermogram, and/or thermal gravimetric analysis (TGA) curve, that may be substantially as illustrated by FIGS. 1, 2, 3, and 4, respectively.

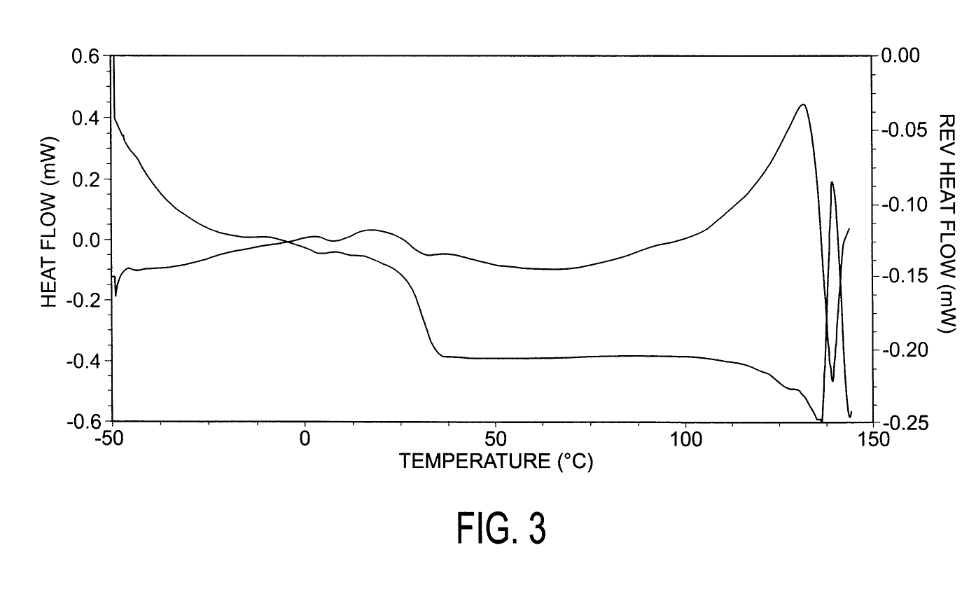

In one variant, the amorphous form of dexlansoprazole prepared according to the processes described in the present application has a glass transition temperature about 31° C. in a differential scanning calorimetric (DSC) thermogram as illustrated in FIG. 3, which is an indication of a relatively high stability of a drug for pharmaceutical use.

Differential scanning calorimetric analyses reported herein were carried out by a conventional modulated differential scanning calorimetric method on a DSC Q1000 V 9.4 Build 287 model from TA Instruments with a ramp of 3° C./minute up to 150° C., with a modulation time of 60 seconds and a modulation temperature of ±1° C. The starting temperature was −50° C. and ending temperature was 150° C.

The X-ray powder diffraction patterns described herein were obtained using a Bruker axs D8 advance diffractometer, equipped with Bragg-Brentano 0:0 goniometer having lynx-eye detector. The radiation was copper Kα-1.

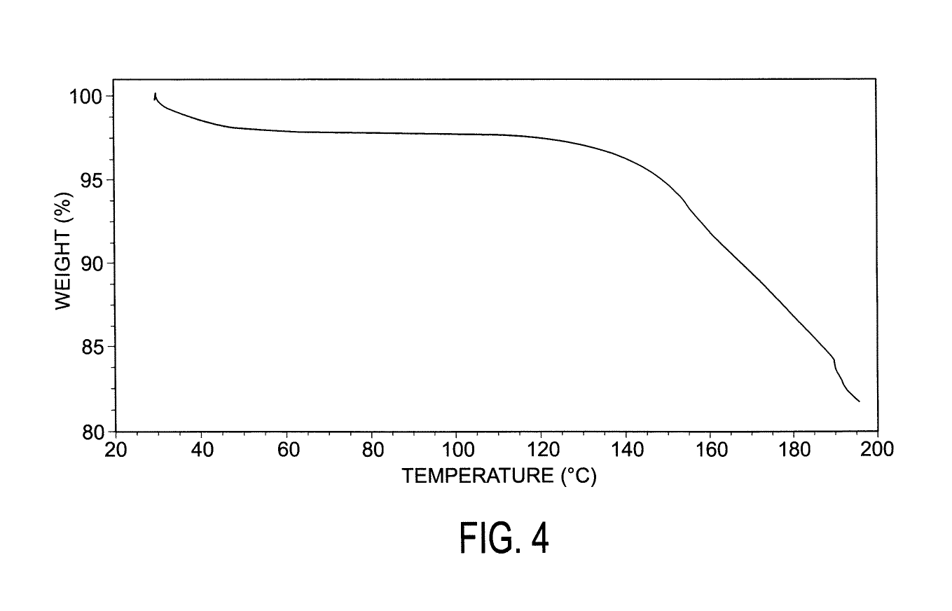

In one variant, an amorphous form of dexlansoprazole prepared according to the process described in the present application has a characteristic TGA curve corresponding to a weight loss of less than about 3% by weight, as illustrated in FIG. 4.

In an embodiment, the present application provides dexlansoprazole having a chemical purity greater than about 99%, or greater than about 99.4%, or greater than about 99.6%, or greater than about 99.8%, by weight as measured by HPLC.

In an embodiment, the present application provides dexlansoprazole having an enantiomeric purity greater than about 99%, or greater than about 99.2%, or greater than about 99.4%, or greater than about 99.6%, or greater than about 99.8%, or greater than about 99.9%, by weight as measured by HPLC.

Dexlansoprazole according to the present application may be substantially free of one or more of the corresponding impurities as measured by HPLC.

“Substantially free of one or more of the corresponding impurities” as used herein, unless otherwise defined refers to the compound that contains less than about 2%, or less than about 1%, or less than about 0.5%, or less than about 0.3%, or less than about 0.1%, or less than about 0.05%, by weight, of each individual impurity including, without limitation, a nitro sulphide impurity of formula (Ib), a nitro sulphone impurity of formula (Ic), a lansoprazole sulphide impurity of formula (Id), a lansoprazole sulphone impurity of formula (Ie), an N-alkylated impurity of formula (If), a chloro sulphide impurity of formula (Ig), a chloro suphone impurity of formula (Ih), a nitro suphoxide impurity of formula (II), a chloro sulphoxide impurity of formula (ID, a 2-mercaptobenzimidazole of formula (Im), a hydroxylmethyl impurity of formula (In), a sulfonyloxy impurity of formula (Io), an impurity at RRT 1.98 having mass number m/z 467, or unwanted enantiomers, or any other possible residual impurity; and that contains a total amount of impurities of less than about 2%, or less than about 1%, or less than about 0.5%, or less than about 0.3%, or less than about 0.1%, or less than about 0.05%, by weight as measured by HPLC.

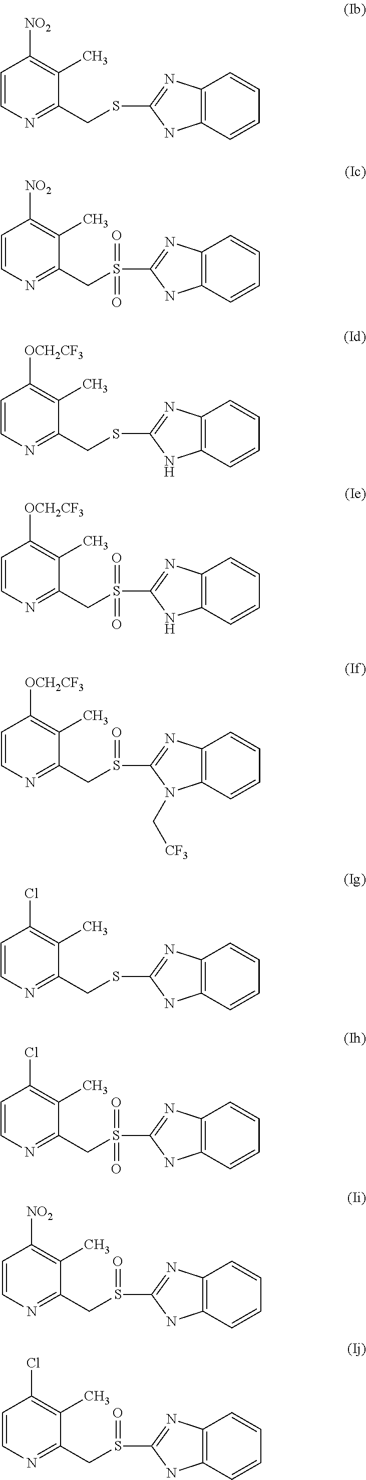

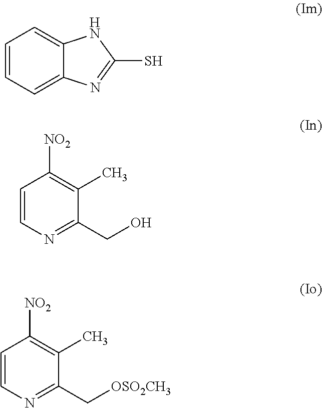

A high performance liquid chromatography (HPLC) method for measuring the chemical purity of dexlansoprazole or a compound of formula (VII) of the present application, and for determination of their corresponding impurities, involves the use of a YMC-PAK ODS-A 100×4.6, 3 μm or equivalent column. Other parameters of the method are as shown in Table 3.

TABLE 3

| Flow | 1.0 mL/minute | | |
|---|---|---|---|
| Elution | Gradient | | |
| Wavelength | 285 nm | | |
| Injection volume | 15 μL | | |
| Oven temperature | Ambient | | |
| Mobile phase | Mobile phase A: water. | | |
| preparation | Mobile phase B: degassed | | |
| | mixture of acetonitrile, water, | | |
| | and triethylamine in the volume | | |
| | ratio of 160:40:1, having pH | | |
| | adjusted to 7.0 with | | |
| | orthophosphoric acid. | | |
| Diluent | Mix one volume of triethylamine | | |
| | with 60 volumes of water, adjust | | |
| | to pH 10.5 using | | |
| | orthophosphoric acid, then mix | | |
| | the solution obtained with 40 | | |
| | volumes of acetonitrile. | | |
| Sample | 1.0 mg/ml, in diluent | | |
| concentration | | | |
| Gradient | Time | | |
| programme | (minutes) | % A | % B |
| | 0 | 75 | 25 |
| | 5 | 75 | 25 |
| | 30 | 40 | 60 |
| | 40 | 20 | 80 |
| | 50 | 20 | 80 |
| | 52 | 75 | 25 |
| | 60 | 75 | 25 |

A high performance liquid chromatography method useful for measuring the enantiomeric purity of dexlansoprazole or a compound of formula (VII) of the present application involves the use of a Chiralpak-IC, 250×4.6 mm or equivalent column. Other parameters of the method are as shown in Table 4.

TABLE 4

| | |
|---|---|
| Flow | 1.0 mL/minute |
| Elution | Gradient |
| Wavelength | 285 nm |
| Injection volume | 10 μL |
| Oven temperature | Ambient |
| Mobile phase | Acetonitrile, triethylamine, |
| preparation | and diethyltartrate in a |
| | 1000:0.5:1 volume ratio. |
| Diluent | Mobile phase. |
| Sample | 1.0 mg/ml, in diluent. |
| concentration | |
| Run time | 25 minutes |

A high performance liquid chromatography (HPLC) method for measuring the impurity at RRT 1.98, and having a mass number of m/z 467, in the present application involves the use of a YMC-PRO C-18 100×4.6 mm, 3 microns or equivalent column. Other parameters of the method are as shown in Table 5.

TABLE 5

| | |
|---|---|
| Flow | 0.8 mL/minute. |
| Elution | Gradient. |
| Wavelength | 285 nm. |
| Injection volume | 15 μL. |
| Oven temperature | Ambient. |
| Mobile phase | Mobile phase A: degassed |
| preparation | water. |
| | Mobile phase B: filtered and |
| | degassed mixture of |
| | acetonitrile, water, and |
| | triethylamine in the ratio of |
| | 160:40:1 (v/v), pH adjusted to |
| | 6.2 with orthophosphoric acid. |
| Diluent | Mix one volume of triethylamine |
| | with 60 volumes of water, adjust |
| | to pH 10.5 using |
| | orthophosphoric acid, then mix |
| | the solution obtained with 40 |
| | volumes of acetonitrile. |
| Sample | 1 mg/ml in diluent. |
| concentration | |

The present application also provides physical characteristics such as particle size distributions, bulk densities, Hausner ratios, and specific surface areas of dexlansoprazole, which are suitable for pharmaceutical use.

In an embodiment, the present application provides dexlansoprazole having a 10^{th} volume percentile particle size (D₁₀) of less than about 5 μm, a 50^{th} volume percentile particle size (D₅₀) of less than about 15 μm, a 90^{th} volume percentile particle size (D₉₀) of less than about 50 μm, and/or any combinations thereof.

“10^{th} volume percentile” as used herein, unless otherwise defined refers to the size of particles, below which 10% of the measured sample volume lies; “50^{th} volume percentile” as used herein, unless otherwise defined refers to the size of particles, below which 50% of the measured samples volume lies, and “90^{th} volume percentile” as used herein, unless otherwise defined refers to the size of particles, below which 90% of the measured samples volume lies.

Particle size distributions of dexlansoprazole particles may be measured with a Jayant Test Siever (e.g., using mesh number 60, mesh opening 250 μm). Particle size distributions of dexlansoprazole particles may also be measured using light scattering equipment such as a Malvern Master Sizer 2000 (helium neon laser source, dexlansoprazole suspended in light liquid paraffin, size range: 0.02 μm to 2000 μm). Other techniques are also useful.

An amorphous form of dexlansoprazole of the present application has desirable characteristics such as being stable to colour change, making it suitable for pharmaceutical use.

As used herein “stable to colour change” unless otherwise defined refers to an amorphous form of dexlansoprazole that shows no change in colour upon storage. In one variant stable amorphous dexlansoprazole of the present application may be characterized by its white to off-white colour, which does not change upon storage at temperatures of about 2° C. to about 8° C.

In an embodiment, the present application provides amorphous dexlansoprazole having a water content of less than about 5%, or less than about 3%, or less than about 2%, or less than about 1%, or less than about 0.5%, by weight as measured by the Karl Fischer method. Water content is expressed in wt/wt, which refers to percentage weight of water with respect to the total weight of the sample when analyzed by Karl Fischer method. Amorphous dexlansoprazole containing the described water content is observed to be less hygroscopic and thus more stable and desirable. Further, if more than about 5% wt/wt water content is present in amorphous dexlansoprazole, crystallinity tends to develop during storage, thus making the amorphous dexlansoprazole unstable. Thus, the described range of water content is useful for enhancing the stability of amorphous dexlansoprazole.

In an embodiment, the present application provides amorphous dexlansoprazole substantially free of residual organic solvents.

“Substantially free of residual organic solvents”, as used herein, unless otherwise defined refers to the compound that contains residual solvent content of less than about 2%, or less than about 1%, or less than about 0.5%, or less than about 0.1%, or less than about 0.05%, by weight as measured by gas chromatography (GC).

In one variant, amorphous dexlansoprazole of the present application may contain less than about 20,000 ppm (parts per million), or less than about 10,000 ppm, or less than about 5,000 ppm, or less than about 1,000 ppm, or less than about 500 ppm, or less than about 300 ppm, or less than about 200 ppm, or less than about 100 ppm, or less than about 50 ppm, or less than about 10 ppm, of individual residual organic solvents.

In another embodiment, the present application provides a process for the preparation of amorphous dexlansoprazole, substantially free of residual organic solvents, which includes one or more of the following steps:

a) micronizing dexlansoprazole; and

b) drying the product obtained from step a) to provide an amorphous form of dexlansoprazole substantially free of residual organic solvents.

Step a) involves micronization of dexlansoprazole.

Step a) may be performed before or after drying of wet dexlansoprazole. Techniques that may be used for micronization include, without limitation, milling with ball, roller, hammer and jet mills.

Step b) involves optionally drying the resultant product obtained from step a).

Drying may be suitably carried out in a tray dryer, vacuum oven, rotational device such as a Buchi Rotavapor, air oven, fluidized bed dryer, spin flash dryer, flash dryer and the like. The drying may be carried out at temperatures less than about 200° C., or less than about 150° C., or less than about 100° C., or less than about 60° C., or less than about 40° C., or less than about 20° C., or less than about 0° C., or less than about −20° C., or any other suitable temperature, at atmospheric pressure or under reduced pressure. The drying may be carried out for any time period that produces a desired quality, such as from about 15 minutes to several hours.

In an embodiment, the present application provides a process for packaging and storing of amorphous dexlansoprazole with increased stability and shelf life, which includes one or more of the following steps:

a) placing dexlansoprazole in a sealed container under an inert atmosphere;

b) placing the sealed container and moisture adsorbent in a second sealed container;

c) placing the second sealed container in a triple laminated bag followed by sealing; and

d) placing the triple laminated bag in a HDPE container and storing in a controlled environment at about 2-8° C.

Step a) involves placing dexlansoprazole in a sealed container under an inert atmosphere.

The inert atmosphere may be provided using any of the inert gases such as nitrogen, argon, and the like. The gas should not react with dexlansoprazole and should be substantially free from moisture.

The inert atmosphere may be provided to the compound which is kept in a polythene bag, or has been stored in a more rigid container. The bag or container which is used to provide the inert atmosphere to dexlansoprazole is sealed air-tight after providing the inert atmosphere.

If the container which is used to provide the inert atmosphere to dexlansoprazole is transparent and exposes the product to light, then it can be covered using a non-transparent material.

Step b) involves placing the sealed container and moisture adsorbent in a second sealed container.

The moisture adsorbent is included to absorb any moisture which enters the packaging. Suitable moisture adsorbents which can be used in the present application include, but are not limited to, molecular sieve zeolites, high silica zeolites, having a high silica/alumina ratio of 25 or more, such as ZSM-5 (made by Mobil Oil Co., silica/alumina ratio of 400), silicalite, USY (Ultra Stable Y type zeolite, by PQ Corp., silica/alumina ratio of 78), mordenite and the like, a low silica system zeolite such as Ca—X type zeolite, Na—X type zeolite, silica super fine granulated particles (for example, particles having an average particle size of 1.5 mm, obtained by granulating the silica super fine particle having a size of 0.1 μm or less), silica gel, γ-alumina, and the like.

Step c) involves placing a second bag or container in a triple laminated bag followed by sealing.

The packaging containing the compound and moisture adsorbents is kept in a triple laminated bag, having layers of polyethylene terephthalate film, aluminum foil, and linear low-density polyethylene film. The triple laminated bag provides protection to the contents from oxygen, water vapor, light and other contaminants.

Optionally, an additional moisture adsorbent is put into the triple laminated bag as an additional precaution to adsorb any moisture which enters it.

The triple laminated bag can be heat sealed to prevent the entry of any contaminants. The heat sealing can be done using a vacuum nitrogen sealer (VNS) for effective sealing.

Step d) involves placing the triple laminated bag in a HDPE container and storing in a controlled environment at about 2-8° C.

It has been found that the above packaging and storage process provides amorphous dexlansoprazole, which is stable during storage and does not change its colour, does not undergo agglomeration and thus results in stable amorphous dexlansoprazole.

In one variant, the present application provides an amorphous form of dexlansoprazole, which is stable to storage. It has been observed that the temperature that is used for storing amorphous dexlansoprazole of the present application plays a role in the formation of the impurity at RRT 1.98 to an undesired level for a pharmaceutical product.

“Stable to storage” as used herein, unless otherwise defined, refers to the compound that may be stable to the increase in levels of an impurity including, without limitation, the impurity at RRT 1.98 or any other corresponding impurity; to concentrations greater than about 0.2%, or greater than about 0.15%, or greater than about 0.1%, or greater than about 0.05%, by weight as measured by HPLC.

In an embodiment, the present application provides dexlansoprazole having having specific surface areas more than about 0.5 m²/g, or more than about 1 m²/g, or more than about 2 m²/g, or more than about 3 m²/g, or more than about 5 m²/g. “Specific surface area” as used herein, unless otherwise defined refers to the total particle surface of 1 gram of particles of a given material per square meter of particle surface area.

The specific surface area of dexlansoprazole of the present application has been measured by a BET (Brunauer, Emmett and Teller) specific surface method using a Micromeritics Gemini surface area analyzer, model 2365. Samples for analysis were degassed at 40° C. under reduced pressure and the determination of the adsorption of nitrogen gas at 77° K was measured for relative pressure in the range of 0.05-0.3.

In an embodiment, the present application provides dexlansoprazole having bulk densities less than about 1 g/ml. Bulk density has been determined using Test 616 “Bulk Density and Tapped Density,” in United States Pharmacopoeia 29, United States Pharmacopeial Convention, Inc., Rockville, Md., 2005, in method 2.

The amorphous form of dexlansopraozole of the present application is stable and is suitable for preparing pharmaceutical formulations for pharmaceutical use.

An aspect of the present application provides compositions comprising dexlansoprazole substantially free of one or more of the corresponding impurities as measured by HPLC. The compositions comprise dexlansoprazole that contains less than about 2%, or less than about 1%, or less than about 0.5%, or less than about 0.3%, or less than about 0.1%, or less than about 0.05%, by weight, of each individual impurity including, without limitation, a nitro sulphide impurity of formula (Ib), a nitro sulphone impurity of formula (Ic), a lansoprazole sulphide impurity of formula (Id), a lansoprazole sulphone impurity of formula (Ie), a N-alkylated impurity of formula (If), a chloro sulphide impurity of formula (Ig), a chloro suphone impurity of formula (Ih), a nitro suphoxide impurity of formula (II), a chloro sulphoxide impurity of formula (Ij), 2-mercaptobenzimidazole of formula (Im), a hydroxylmethyl impurity of formula (In), a sulfonyloxy impurity of formula (Io), an impurity at RRT 1.98 having a mass number m/z 467, or unwanted enantiomers, or any other possible residual impurity, and that contains a total amount of impurities of less than about 2%, or less than about 1%, or less than about 0.5%, or less than about 0.3%, or less than about 0.1%, or less than about 0.05%, by weight as measured by HPLC.

An aspect of the present application provides pharmaceutical compositions comprising dexlansoprazole having a particle size distribution wherein a 10^{th} volume percentile particle size (D₁₀) is less than about 5 μm, a 50^{th} volume percentile particle size (D₅₀) is less than about 15 μm, a 90^{th} volume percentile particle size (D₉₀) is less than about 50 μm, and/or any combination thereof, together with one or more pharmaceutically acceptable excipients.

An aspect of the present application provides pharmaceutical compositions comprising dexlansoprazole having having a specific surface area more than about 0.5 m²/g, or more than about 1 m²/g, or more than about 2 m²/g, or more than about 3 m²/g, or more than about 5 m²/g, together with one or more pharmaceutically acceptable excipients.

An aspect of the present application provides pharmaceutical compositions comprising dexlansoprazole having bulk density less than about 1 g/ml, together with one or more pharmaceutically acceptable excipients.

In an embodiment, the present application provides a pharmaceutical composition comprising amorphous dexlansoprazole together with one or more pharmaceutically acceptable excipients.

In an embodiment, the present application provides a process for the preparation of crystalline dexlansoprazole, which includes one or more of the following steps:

a) providing a mixture comprising a salt of dexlansoprazole;

b) adjusting the pH of the reaction mixture obtained from step a) with an acid to obtain dexlansoprazole; and

c) isolating crystalline dexlansoprazole from the reaction mixture obtained in step b).

Step a) involves providing a reaction mixture comprising a salt of dexlansoprazole.

The mixture comprising a salt of dexlansoprazole in step a) may be obtained directly from a reaction mixture that is obtained in the course of its manufacture. For example, it may be obtained by a process as described in the present application.

Step b) involves adjusting the pH of the reaction mixture obtained form step a) with an acid.

An acid may be used to adjust the pH in step b). Suitable acids that may be used in step b) include but are not limited to: organic acids such as acetic acid, formic acid, trifluoroacetic acid, chloroacetic acid, propionic acid, butanoic acid, isobutyric acid, valeric acid, isovaleric acid, benzoic acid, salicylic acid, phthalic acid, p-toluene sulphonic acid, o-toluene sulphonic acid, benzene sulphonic acid, methane sulphonic acid, ethane sulphonic acid and the like; ion exchange resins such as resins bound to acids such as p-toluene sulphonic acid, sulphuric acid, phosphoric acid, styrene-divinylbenzenesulfonic acid and the like; chelated resins; neutral resins; and any other suitable reagent, which may bring the pH in step b) to the desired level without affecting the quality of dexlansoprazole.

In one variant, pH and the temperature conditions at which pH may be adjusted in step b) play a role in producing the desired quality and yield of dexlansoprazole.

pH may be adjusted to about 7 to about 9, or any other suitable pH, which may dissociate the salt that is present in the reaction mixture in step a).

The temperatures at which pH may be adjusted are less than about 40° C., or less than about 30° C., or less than about 20° C., or less than about 10° C., or less than about 5° C., or less than about 0° C., or any other suitable temperatures that do not affect the quality and yield of dexlansoprazole.

Optionally, any insoluble solids or particles may be removed from the mixture comprising a salt of dexlansoprazole in step a), before pH adjustment of the reaction mixture in step b). Suitable techniques that may be used to remove insoluble solids or particles include methods such as decantation, centrifugation, gravity filtration, suction filtration or any other suitable technique for the removal of solids.

Optionally, the resulting solution that may be obtained after removal of insoluble solids or particles before step b) may optionally be treated with carbon, flux-calcined diatomaceous earth (Hyflow) or any other suitable material to remove colour and/or to improve clarity of the solution, before pH adjustment of reaction mixture in step b).

Optionally, a water miscible solvent may be added to the mixture, before or after pH adjustment. The water miscible solvents that may be added include but are not limited to: alcohols such as methanol, ethanol, 1-propanol, and the like; ketones such as acetone, and the like; ethers such as tetrahydrofuran, 1,4-dioxane, and the like; nitriles such as acetonitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

Step c) involves isolating crystalline dexlansoprazole from the mixture obtained in step b).

Isolation of crystalline dexlansoprazole in step c) may involve methods including removal of solvent, cooling, concentrating the reaction mass, adding an anti-solvent, extraction with a solvent and the like. Stirring or other alternate methods such as shaking, agitation and the like, may also be employed for the said isolation. The suitable temperatures for isolation may be less than about 100° C., or less than about 60° C., or less than about 40° C., or less than about 20° C., or less than about 10° C., or less than about 5° C., or less than about 0° C., or less than about −10° C., or less than about −20° C., or any other suitable temperatures. Suitable times for isolation may be less than about 5 hours, or less than about 3 hours, or less than about 2 hours, or less than about 1 hour, or longer times may be used. However, the exact temperatures and times required for complete isolation may be readily determined by a person skilled in the art and will also depend on parameters such as concentration and temperature of the solution or slurry.

The crystalline dexlansoprazole may be recovered by methods including decantation, centrifugation, gravity filtration, suction filtration or any other technique for the recovery of solids. The crystalline dexlansoprazole thus isolated may carry some amount of occluded mother liquor and may have higher than desired levels of impurities. If desired, these crystals may be washed with a solvent or a mixture of solvents to wash out the impurities.

The recovered solid may be optionally further dried. Drying may be carried out in a tray dryer, vacuum oven, air oven, fluidized bed dryer, spin flash dryer, flash dryer and the like. The drying may be carried out at temperatures less than about 150° C., or less than about 120° C., or less than about 100° C., or less than about 80° C., or less than about 60° C., or less than about 50° C., or less than about 30° C., or any other suitable temperatures as long as the dexlansoprazole is not degraded in quality, at atmospheric pressure or under a reduced pressure. The drying may be carried out for any desired times until the required quality is achieved. For example, it may vary from about 1 to about 12 hours, or longer.

In an embodiment, the present application provides a solid dispersion containing amorphous dexlansoprazole, together with one or more pharmaceutically acceptable carriers, with the proviso that the carrier is not a base.

Solid dispersions containing amorphous dexlansoprazole, together with a pharmaceutically acceptable carrier, provide a product with desired characteristics such as stability and are suitable for preparing pharmaceutical formulations for pharmaceutical use.

In an aspect, the present application provides a process for preparing solid dispersions containing amorphous dexlansoprazole together with one or more pharmaceutically acceptable carriers, with the proviso that the carrier is not a base; which includes one or more of the following steps:

a) providing a solution of dexlansoprazole in combination with at least one pharmaceutically acceptable carrier, with the proviso that the carrier is not a base, in a suitable solvent or mixture of solvents;

b) isolating a solid dispersion of amorphous dexlansoprazole together with one or more pharmaceutically acceptable carriers.

Step a) involves providing a solution of dexlansoprazole in combination with at least one pharmaceutically acceptable carrier, with the proviso that the carrier is not a base.

Step a) may involve forming a solution of dexlansoprazole together with one or more pharmaceutically acceptable carriers. In embodiments, a carrier enhances stability of the amorphous solid upon removal of solvent.

Providing the solution in step a) includes:

i) direct use of a reaction mixture containing dexlansoprazole that is obtained in the course of its manufacture, if desired, after addition of one or more pharmaceutically acceptable carriers; or

ii) dissolution of dexlansoprazole in a suitable solvent, either alone or in combination with one or more pharmaceutically acceptable carriers.

Any physical form of dexlansoprazole, such as crystalline, amorphous or their mixtures may be utilized for providing a solution in step a).

Pharmaceutically acceptable carriers that may be used in step a) include, but are not limited to: pharmaceutical hydrophilic carriers such as polyvinylpyrrolidones (homopolymers or copolymers of N-vinylpyrrolidone), gums, cellulose derivatives (including hydroxypropyl methylcelluloses, hydroxypropyl celluloses and others), polymers of carboxymethyl cellulose, cyclodextrins, gelatins, hypromellose phthalates, polyhydric alcohols, polyethylene glycols, polyethylene oxides, polyoxyethylene derivatives, polyvinyl alcohols, propylene glycol derivatives and the like; and organic amines such as alkyl amines (primary, secondary, and tertiary), aromatic amines, alicyclic amines, cyclic amines, aralkyl amines, hydroxylamine or its derivatives, hydrazine or its derivatives, and guanidine or its derivatives. The use of mixtures of more than one of the carriers to provide desired release profiles or for the enhancement of stability is within the scope of this application. Also, all viscosity grades, molecular weights, commercially available products, their copolymers, and mixtures are all within the scope of this application without limitation.

Suitable solvents that may be used in step a) include but are not limited to: water; alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol and the like; ketones such as acetone, butanone; 2-pentanone, 3-pentanone, methyl butyl ketone, methyl isobutyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; chlorinated hydrocarbons such as dichloromethane, chloroform, 1,1,2-trichloroethane, 1,2-dichloroethene and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; and mixtures thereof.

The dissolution temperatures may be less than about 150° C., or less than about 100° C., or less than about 60° C., or less than about 40° C., or any other suitable temperatures depending on the solvent used for dissolution. Any other temperatures are also acceptable as long as a clear solution is obtained without affecting the quality of dexlansoprazole.

The solution may optionally be treated with materials such as carbon, Hyflow or any other suitable material to remove colour or to improve clarity of the solution.

Optionally, the solution obtained above may be filtered to remove any insoluble particles. The insoluble particles may be removed suitably by techniques known in the art such as by filtration, centrifugation, decantation or any other suitable technique. The solution may be filtered by passing through paper, glass fiber, or other membrane material, or a bed of a clarifying agent such as celite or Hyflow. Depending upon the equipment used and the concentration and temperature of the solution, the filtration apparatus may need to be preheated to avoid premature crystallization.

Step b) involves isolation of a solid dispersion of amorphous dexlansoprazole together with one or more pharmaceutically acceptable carriers from the solution of step a).

In one variant, the isolation may be effected by removing solvent.

Suitable techniques which may be used for the removal of solvent include using a rotational distillation device such as a Buchi Rotavapor, spray drying, agitated thin film drying (“ATFD”), freeze drying (lyophilization) and the like or any other suitable techniques.

The solvent may be removed, optionally under reduced pressure, at temperatures of less than about 200° C., or less than about 150° C., or less than about 100° C., or less than about 60° C., or less than about 40° C., or less than about 20° C., or less than about 0° C., or less than about −20° C., or less than about −40° C., or less than about −60° C., or less than about −80° C., or any other suitable temperatures.

Freeze drying (lyophilization) may be carried out by freezing a solution containing dexlansoprazole at low temperatures required to freeze the solution, and reducing the pressure as required to remove the solvent from the frozen solution. Temperatures that may be required freeze the solution depending on the solvent selected to make the solution of dexlansoprazole may range from about −80° C. to about 0° C., or up to about 40° C. Temperatures that may be required to remove the solvent from the frozen solution may be less than about 20° C., or less than about 0° C., or less than about −20° C., or less than about −40° C., or less than about −60° C., or less than about −80° C., or any other suitable temperatures.

Alternatively, isolation may be effected by adding a suitable anti-solvent to the solution obtained in step a), optionally after concentrating the solution obtained in step a). Suitable anti-solvents that may be used include but are not limited to: ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; aromatic hydrocarbons such as toluene, xylenes; chlorobenzene; tetraline; and the like; and mixtures thereof.

The dispersion obtained from step b) may be collected using techniques such as by scraping, or by shaking the container, or other techniques specific to the equipment used.

Step b) optionally further includes drying of the product obtained from step b) to afford a solid dispersion of amorphous dexlansoprazole together with a pharmaceutically acceptable organic excipient.

The product obtained in step b) may optionally be further dried. Drying may be suitably carried out in a tray dryer, vacuum oven, Buchi Rotavapor, air oven, fluidized bed dryer, spin flash dryer, flash dryer and the like. The drying may be carried out at temperatures of less than about 200° C., or less than about 150° C., or less than about 100° C., or less than about 60° C., or less than about 40° C., or less than about 20° C., or less than about 0° C., or less than about −20° C., or any other suitable temperatures, at atmospheric pressure or under reduced pressure. The drying may be carried out for any time periods desired for obtaining a particular product quality, such as from about 15 minutes to several hours.

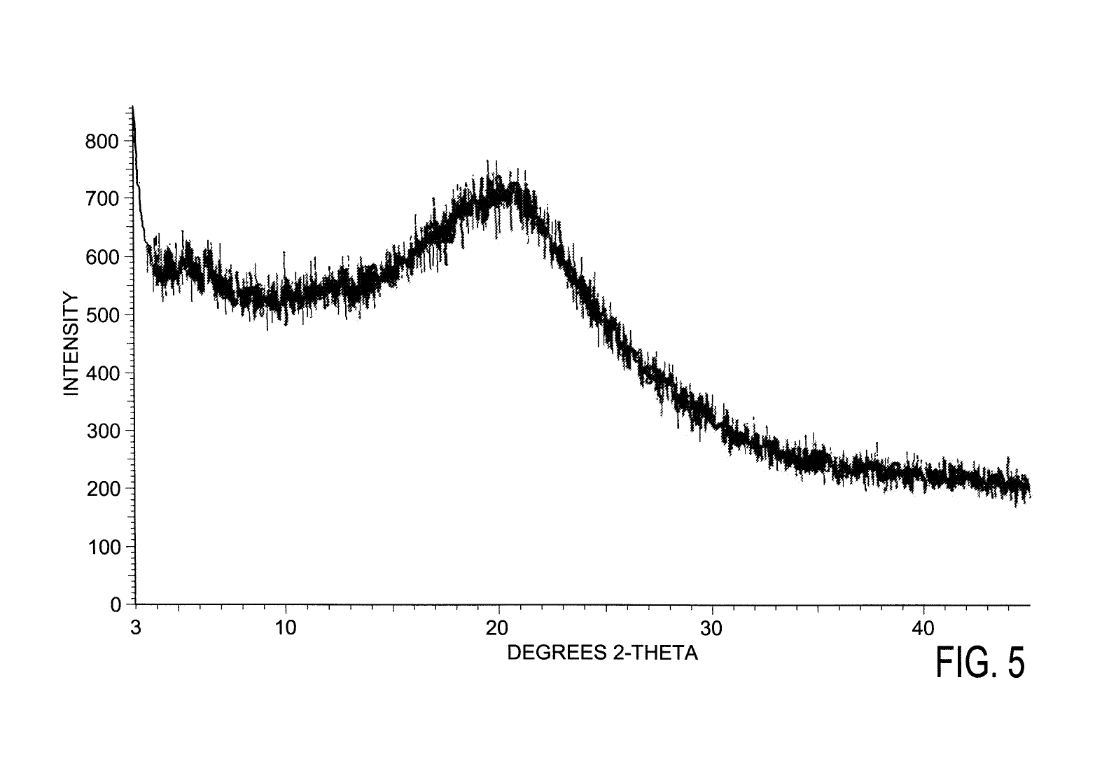

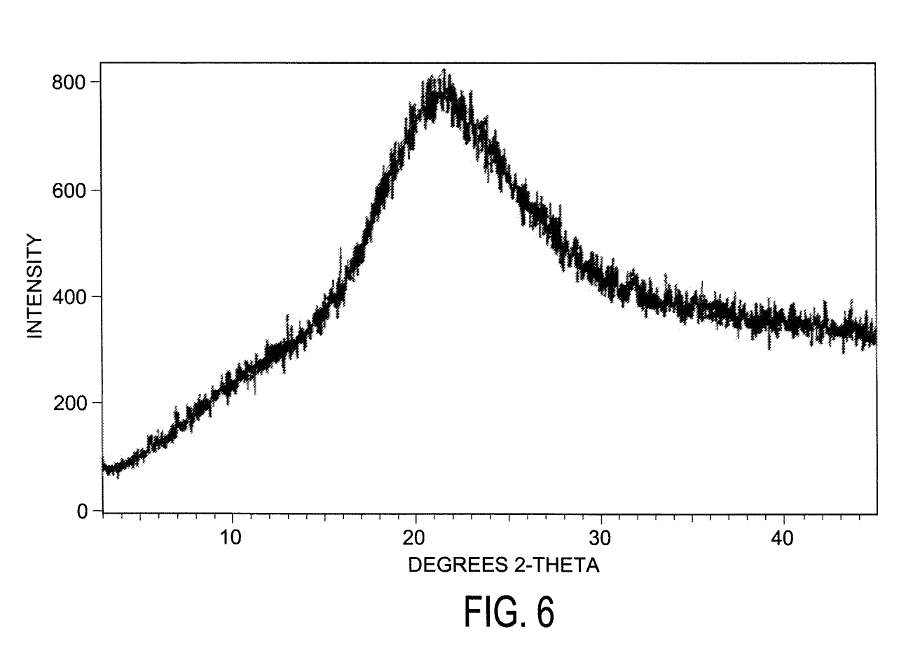

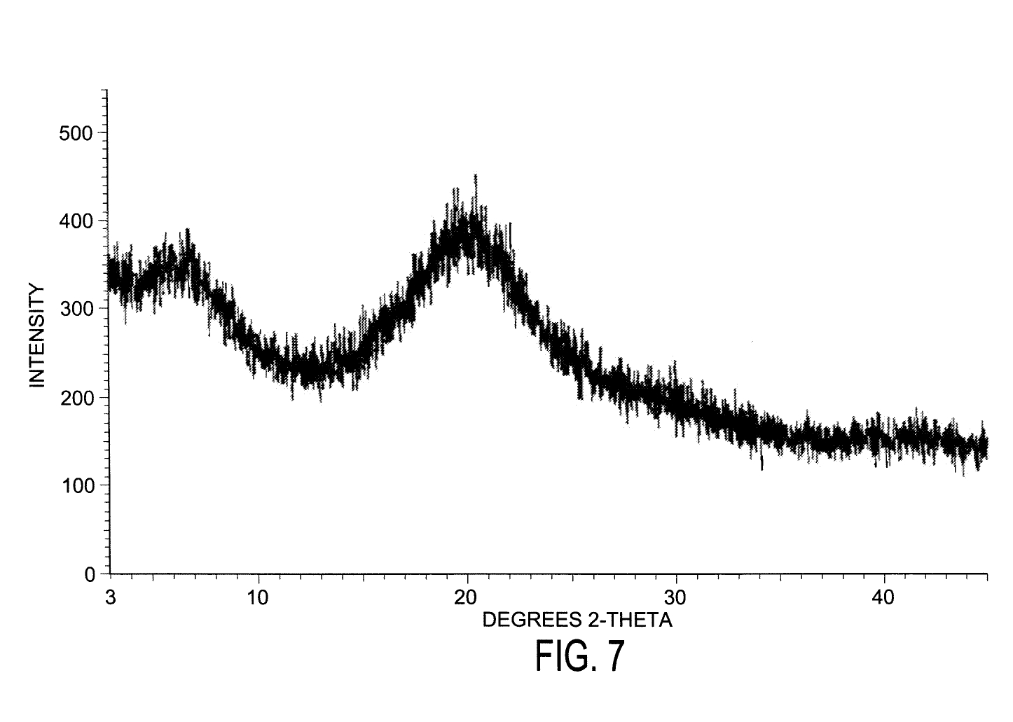

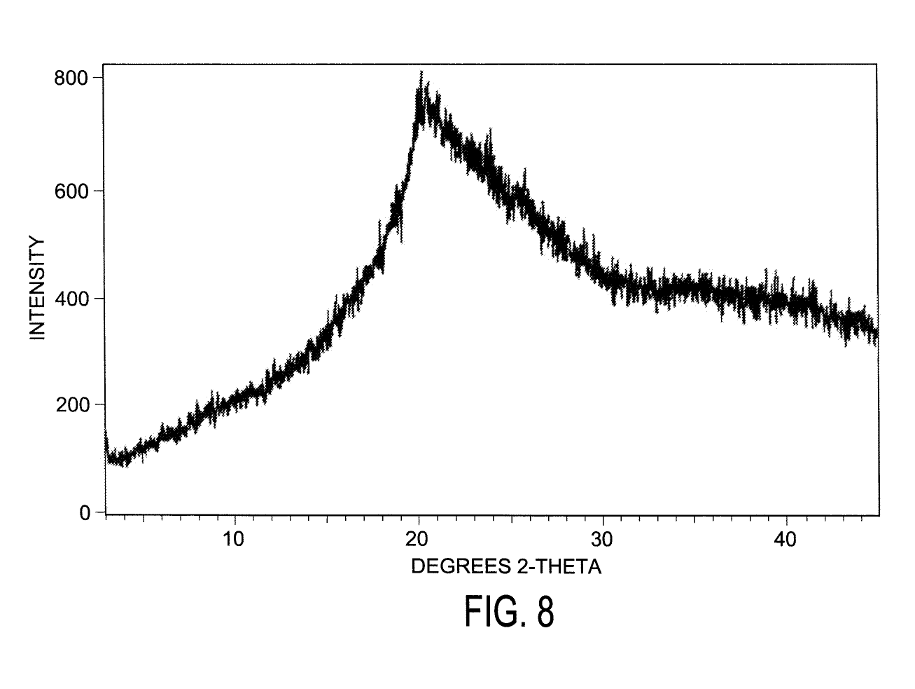

Examples of amorphous solid dispersions of dexlansoprazole together with a pharmaceutically acceptable carrier obtained using the above process are characterized by their powder X-ray diffraction (“PXRD”) patterns substantially as illustrated by FIGS. 5, 6, 7 and 8, respectively.

The solid dispersions differ from physical mixtures of amorphous dexlansoprazole and one or more pharmaceutically acceptable carriers, in that individual particles of the components cannot be distinguished using techniques such as optical microscopy. In instances, the solid dispersions contain the components on a molecular level, such as in the nature of solid solutions.

In an aspect, the present application also provides pharmaceutical formulations comprising solid dispersions of amorphous dexlansoprazole together with one or more pharmaceutically acceptable excipients.

A solid dispersion of dexlansoprazole together with one or more pharmaceutically acceptable excipients of the present application may be further formulated as: solid oral dosage forms such as, but not limited to: powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the forms of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using techniques such as direct blending, dry granulation, wet granulation, and extrusion and spheronization. Compositions may be presented as uncoated, film coated, sugar coated, powder coated, enteric coated, and modified release coated. Compositions of the present application may further comprise one or more pharmaceutically acceptable excipients.

Pharmaceutically acceptable excipients that are useful in the present application include, but are not limited to: diluents such as starches, pregelatinized starches, lactose, powdered celluloses, microcrystalline celluloses, dicalcium phosphate, tricalcium phosphate, mannitol, sorbitol, sugar and the like; binders such as acacia, guar gum, tragacanth, gelatin, polyvinylpyrrolidones, hydroxypropyl celluloses, hydroxypropyl methylcelluloses, pregelatinized starches and the like; disintegrants such as starches, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants, and the like.

Certain specific aspects and embodiments of the present application will be explained in more detail with reference to the following examples, which are provided for purposes of illustration only and should not be construed as limiting the scope of the invention in any manner.

### Example 1

### Preparation of 2-[(R)-[(4-nitro-3-methyl-2-pyridinyl)methyl]sulfinyl]-1H-benzimidazole

2-[[(4-nitro-3-methyl-2-pyridinyl)methyl]thio]-1H-benzimidazole (10.2 g) and toluene (300 mL) were charged into a round bottom flask under a nitrogen atmosphere and stirred for 5-10 minutes at 25-35° C. Water (0.09 mL) and (+)-diethyltartrate (2.5 mL) were charged and stirred for 5-10 minutes at 25-35° C. The mixture was heated to 65-70° C. and maintained for 30 minutes. Titanium isopropoxide (11.68 mL) was added to the mixture at 65-70° C. and maintained for 1-2 hours. The mixture was cooled to 15-20° C. and diisopropylethylamine (5.73 mL) was added and stirred for 5-10 minutes. The mixture was cooled to 0-5° C. and cumene hydroperoxide (8.22 mL) was added over 20-30 minutes. The reaction mixture was maintained at 0-5° C. for 4-5 hours. The mass was extracted with 12.5% piperidine solution (2×100 mL) and 12.5% aqueous ammonia solution (2×100 mL) and the combined aqueous layer was washed with toluene (2×25 mL). Acetonitrile (60 mL) was added to the aqueous layer and the solution was cooled to 10-15° C. The pH of the solution was adjusted to 8.5-9 with acetic acid (30 mL) at 10-15° C. and the temperature was raised to 25-35° C. The mass was maintained at 25-35° C. for 1-2 hours and the formed solid was filtered and washed with water (50 mL). The solid was dried at 44° C. under reduced pressure to afford 5.1 g of the title compound. Chiral purity by HPLC 95.78%, chemical purity by HPLC 98.98%, moisture content 5.66%.

### Example 2

### Preparation of 2-[(R)-[(4-nitro-3-methyl-2-pyridinyl)methyl]sulfinyl]-1H-benzimidazole

2-[[(4-nitro-3-methyl-2-pyridinyl)methyl]thio]-1H-benzimidazole (10.6 g) and toluene (300 mL) were charged into a round bottom flask, fitted with a Dean-Stark apparatus, and stirred for 5-10 minutes. The mixture was heated to 110° C. and subjected to azeotropic refluxing for 1-2 hours to remove water completely. The mixture was cooled to 70° C. and water (0.36 mL), (+)-diethyltartrate (12.58 mL) and titanium isopropoxide (11.71 mL) were added and stirred at 65-70° C. for 1 hour. The mixture was cooled to 15-25° C. and diisopropylethylamine (5.73 mL) was added, then the mixture was cooled to 0-5° C. Cumene hydroperoxide (10.38 mL) was added at 0-5° C. over 30-45 minutes and the mixture was maintained at 0-5° C. for 4-5 hours. The reaction was quenched with 12.5% piperidine (200 mL) and the organic and aqueous layers were separated. The organic layer was extracted with 12.5% piperidine (200 mL) and 12.5% aqueous ammonia (2×200 mL). The combined aqueous layer was washed with toluene (2×50 mL). Acetonitrile (60 mL) was added to the aqueous layer and the solution was cooled to 10-15° C. The pH of the solution was adjusted to 8.3 to 8.8 with acetic acid (55 mL). The mass was maintained at 25-35° C. for 2-3 hours. The formed solid was filtered and washed with water (100 mL) and dried at 50° C. to afford 6.2 g of the title compound. Chemical purity by HPLC 99.15%, chiral purity by HPLC 98.17%.

### Example 3

### Optical Purification of 2-[(R)-[(4-nitro-3-methyl-2-pyridinyl)methyl]sulfinyl]-1H-benzimidazole

2-[(R)-[(4-nitro-3-methyl-2-pyridinyl)methyl]sulfinyl]-1H-benzimidazole (5.0 g) and acetone (125 mL) were charged into a round bottom flask and stirred for 5-10 minutes. The mixture was heated to 50-55° C. and maintained to dissolve 2-[(R)-[(4-nitro-3-methyl-2-pyridinyl)methyl]sulfinyl]-1H-benzimidazole completely. The solution was cooled to 25-35° C., further cooled to 5-10° C., and maintained for 1-2 hours at 5-10° C., then the formed solid was filtered and washed with acetone (10 mL). The filtrate was evaporated at 40-45° C. under reduced pressure to afford 3.2 g of the title compound. Chiral purity of input material: 86.12%, chiral purity of product by HPLC 99.49%, chemical purity of product by HPLC 98.14%.

### Example 4

### Preparation of 2-[(R)-[(4-chloro-3-methyl-2-pyridinyl)methyl]sulfinyl]-1H-benzimidazole

2-[[(4-chloro-3-methyl-2-pyridinyl)methyl]thio]-1H-benzimidazole (5.0 g), toluene (125 mL) and water (0.132 mL) were charged into a round bottom flask under a nitrogen atmosphere and stirred for 5-10 minutes. (+)-Diethyltartrate (12.18 mL) was added to the mixture and heated to 55-60° C. and maintained for a period of 15-30 minutes. Titanium isopropoxide (10.2 mL) was added and the mixture was maintained at 55-60° C. for 1 hour. The mixture was cooled to 15° C. and diisopropylethylamine (3.0 mL) was added. The mixture was again cooled to 0 to −5° C. and cumene hydroperoxide (5.63 mL) was added over a period of 10-15 minutes, then the mixture was maintained at 0 to −5° C. for 4-5 hours. Isooctane (5.0 mL) and 12.5% aqueous ammonia solution (100 mL) were added and stirred for 10 minutes, and the organic and aqueous layers were separated. The organic layer was washed with 12.5% aqueous ammonia solution (50 mL) and the combined aqueous layer was extracted with toluene (125 mL). The solution was cooled to 10-15° C., pH was adjusted to 7.5-8 using acetic acid solution (60 mL), and the solution was maintained at 10-15° C. The formed solid was filtered and washed with water (125 mL). The solid was dried at 45° C. to afford 3.6 g of the title compound. Chemical purity by HPLC 99.10%, chiral purity by HPLC 97.59%, moisture content: 2.3%.

### Example 5

### Preparation of 2-[(R)-[[3-methyl-4-(2,2,2-trifluoroethoxy)-2-pyridinyl]methyl]sulfinyl]-1H-benzimidazole

Dimethylformamide (28 mL) and 2,2,2-trifluoroethanol (8.86 g) were charged into a round bottom flask and stirred for 5-10 minutes. The mixture was cooled to 15-20° C. and potassium carbonate (12.2 g) was added and stirred for a period of 5-10 minutes. The mixture was heated to 50-55° C. and maintained at 50-55° C. for 45 minutes, then was cooled to 15-20° C. and a solution of 2-[(R)-[(4-nitro-3-methyl-2-pyridinyl)methyl]sulfinyl]-1H-benzimidazole (4.0 g) in dimethylformamide (12 mL) was added and stirred for 5-10 minutes. The mixture was heated to 90-95° C. and maintained for 5-6 hours. The mixture was cooled to 55-60° C. and water (120 mL) and carbon (1.2 g) were added. The mixture was maintained at 55-60° C. for 30-40 minutes, then was filtered and the solid washed with water (40 mL). Acetonitrile (20 ml) was added to the filtrate and the solution was cooled to 10-15° C. The pH of the solution was adjusted to 8.5 to 9 with 10% acetic acid (40 mL) and maintained for 1-2 hours. The formed solid was filtered and washed with water (20 mL) and dried at 44° C. under reduced pressure to afford 2.9 g of the title compound. Chiral purity by HPLC 96.34%, chemical purity by HPLC 99.1%.

### Example 6

### Preparation of 2-[(R)-[[3-Methyl-4-(2,22-trifluoroethoxy)-2-pyridinyl]methyl]sulfinyl]-1H-benzimidazole

2,2,2-trifluoroethanol (24.5 g) and dimethylacetamide (30 mL) were charged into a round bottom flask and stirred for 5-10 minutes. The mixture was cooled to 10-15° C. and potassium t-butoxide (27.5 g) was added over a period of 10-15 minutes. The mixture was heated to 45-50° C. and maintained for 45-60 minutes. The mixture was cooled to 25-35° C. and a solution of 2-[(R)-[(4-chloro-3-methyl-2-pyridinyl)methyl]sulfinyl]-1H-benzimidazole (15.0 g) in dimethylacetamide (45 mL) was added and stirred for 5-10 minutes. The mixture was heated to 55-60° C. and maintained for 9-10 hours. The mixture was cooled to 5-10° C. and water (100 mL) was added and stirred for 5-10 minutes. The pH was adjusted to 8 to 8.5 with 10% acetic acid solution (70 mL) and maintained at 5-10° C. for 1-2 hours. The formed solid was filtered and washed with water (30 mL) and dried at 25-35° C. to afford 16.6 g of the title compound. Chemical purity by HPLC 86.59%.

### Example 7

### Preparation of Dexlansoprazole

2-[[[3-methyl-4-(2,2,2-trifluoroethoxy)-2-pyridinyl]methyl]thio]-1H-benzimidazole (50.0 g) and toluene (1.25 L) were charged into a vessel and stirred for about 10 minutes. (+)-Diethyltartrate (62.0 mL) and water (0.61 mL) were added and heated to 58° C. The mixture was maintained at 58° C. for 15 minutes. Titanium isopropoxide (42.0 mL) was added and stirred for 1 hour at 58° C. The mixture was cooled to 15° C. and diisopropylethylamine (25.0 mL) was added. Cumene hydroperoxide (26.9 ml) was added at −2° C. over a period of 10 minutes and the mixture was maintained at −10° C. for 3 hours, 30 minutes. 30% sodium thiosulphate solution (180 mL) was added and the mixture was warmed to room temperature. The mixture was filtered through a Hyflow (flux-calcined diatomaceous earth) bed and the layers were separated. The solvent was distilled completely from the organic layer under reduced pressure below 60° C. The residue was cooled to 27° C., n-heptane (500 mL) was added, and the mixture was stirred for 4 hours. The formed solid was filtered, washed with n-heptane (50 ml) and suction dried, to afford 50.0 g of (R)-2-[[[3-methyl-4-(2,2,2-trifluoroethoxy)-2-pyridinyl]methyl]sulfinyl]-1H-benzimidazole.

### Example 8

### Preparation of Amorphous Dexlansoprazole

Dexlansoprazole (0.5 g) and dichloromethane (100 mL) were combined and stirred for about 10 minutes to dissolve dexlansoprazole completely. The solution was filtered and the solvent was distilled completely under reduced pressure at 47° C., to afford 0.5 g of amorphous dexlansoprazole. Chiral purity by HPLC 99.94%.

### Example 9

### Preparation of Amorphous Dexlansoprazole

Dexlansoprazole (0.7 g) and methanol (100 mL) were combined and stirred for about 10 minutes to dissolve dexlansoprazole completely. The solution was filtered and the solvent was distilled completely under reduced pressure at 55° C., to afford 0.7 g of amorphous dexlansoprazole. Chiral purity by HPLC 99.8%.

### Example 10

### Preparation of Amorphous Dexlansoprazole

Dexlansoprazole (0.5 g), acetone (100 mL) and methanol (35 mL) were combined and stirred for 35 minutes to dissolve dexlansoprazole completely. The solution was filtered and the solvent was distilled completely under reduced pressure at 55° C., to afford 0.5 g of amorphous dexlansoprazole. Chiral purity by HPLC 99.91%.

### Example 11

### Preparation of Amorphous Dexlansoprazole

Dexlansoprazole (1.0 g), ethyl acetate (100 mL) and methanol (50 mL) were combined and stirred for about 30 minutes to dissolve dexlansoprazole completely. The solution was filtered and the solvent was distilled completely under reduced pressure at 55° C., to afford 1.0 g of amorphous dexlansoprazole. Chiral purity by HPLC 99.57%.

### Example 12

### Preparation of Solid Dispersion of Amorphous Dexlansoprazole with Povidone

Dexlansoprazole (0.5 g), povidone (0.5 g) and dichloromethane (100 mL) were combined and stirred for about 15 minutes to form a clear solution. The solvent was distilled completely under reduced pressure at 47° C., to afford a solid dispersion of amorphous dexlansoprazole and povidone.

### Example 13

### Preparation of a Solid Dispersion of Amorphous Dexlansoprazole with Hydroxypropyl Methylcellulose

Dexlansoprazole (0.5 g), hydroxypropyl methylcellulose (0.5 g) and dichloromethane (100 mL) were combined and stirred for 15 minutes to produce a suspension. The solvent was distilled completely under reduced pressure at 45° C., to afford 1.0 g of a solid dispersion of amorphous dexlansoprazole with hydroxypropyl methylcellulose.

### Example 14

### Preparation of a Solid Dispersion of Amorphous Dexlansoprazole with Hydroxypropylcellulose

Dexlansoprazole (0.5 g), hydroxypropylcellulose (0.5 g) and dichloromethane (100 mL) were combined and stirred for about 15 minutes. The solvent was distilled completely under reduced pressure at 45° C., to afford 1.0 g of a solid dispersion of amorphous dexlansoprazole with hydroxypropylcellulose.

### Example 15

### Preparation of a Solid Dispersion of Amorphous Dexlansoprazole with Croscarmellose Sodium

Dexlansoprazole (0.5 g), croscarmellose sodium (0.5 g) and dichloromethane (100 mL) were combined and stirred for 15 minutes. The solvent was distilled completely under reduced pressure at 45° C., to afford 1.0 g of a solid dispersion of amorphous dexlansoprazole with croscarmellose.

### Example 16

### Preparation of Amorphous Dexlansoprazole

(A) Dexlansopraozole (10.0 g), dichloromethane (170 mL), and sodium sulphate (17.0 g) were combined and the mixture was stirred to dissolve dexlansoprazole completely. The mixture was filtered and the sodium sulphate cake was washed with dichloromethane (30 mL). The filtrate was evaporated by spray-drying using a BÜCHI MINI Spray Dryer B-290 with BÜCHI Inert Loop B-295 spray-drier, to afford 3.5 g of amorphous dexlansoprazole. Chiral purity by HPLC 96.59%.

Operating parameters for spray dryer: aspirator 70%, feed rate 20% (6 mL/minute), inlet temperature 45° C., N₂ pressure 5.0 Kg/cm².

(B) Dexlansopraozole (20.0 g) and acetone (280 mL) were charged into a round bottom flask and stirred at 25-35° C. for 5-10 minutes to dissolve dexlansoprazole completely. The resulting solution was filtered and the filter washed with acetone (20 mL). The filtrate was evaporated by spray-drying using a BÜCHI MINI Spray Dryer B-290 with BÜCHI Inert Loop B-295 spray-drier, to afford 12.5 g of amorphous dexlansoprazole. Chiral purity by HPLC 96.2%, chemical purity by HPLC 98.13%.

Operating parameters for spray dryer: aspirator 70%, feed rate 40% (12 mL/minute), inlet temperature 60° C., N₂ pressure: 5.0 Kg/cm².

### Example 17

### Preparation of Amorphous Dexlansoprazoleg

Dexlansoprazole (2.0 g) was dissolved in acetonitrile (38 mL) and the solution was filtered through filter paper. The filtrate was placed into a freeze dryer at a temperature of 28° C. and was subjected to freeze drying at −55° C. to 0° C. for about 15-20 hours, to afford 1.6 g of product. Chemical purity by HPLC 98.7%, SOR (specific optical rotation, c=1% w/v in chloroform) [α]_{D}=166.7.

### Example 18

### Preparation of Amorphous Dexlansoprazole

Dexlansoprazole (5 g) and acetone (5 mL) were charged into a round bottom flask and heated to 40° C. to dissolve dexlansoprazole completely. The solution was poured onto ice cubes, mixed, and the formed solid was filtered to afford 0.85 g of product.

### Example 19

### Preparation of Amorphous Dexlansoprazole

(A) Dexlansoprazole (30 g) and dichloromethane (450 mL) were charged into a beaker and stirred for 5 minutes. Sodium sulfate (10 g) was added to the solution and stirred for 5-10 minutes. The solution was filtered and the filtrate was evaporated completely using a Technoforce 0.25 m² agitated thin-film dryer to afford 1.5 g of the product.

ATFD parameters: feed rate: 1 L/hour, temperature 30-35° C., pressure 1-2 torr.

(B) Dexlansoprazole (30 g), methanol (15 mL), and dichloromethane (100 mL) were charged into a beaker and heated to dissolve dexlansoprazole completely. Sodium sulfate (10 g) was added to the solution and stirred for 5-10 minutes. The mixture was filtered and the filtrate was evaporated completely using a Technoforce 0.25 m² agitated thin-film dryer, to afford 1.0 g of the product.

ATFD parameters: feed rate: 2 L/hour, temperature 60-65° C., pressure: 10-15 torr.

### Example 20

### Preparation of Amorphous Dexlansoprazole

Dexlansoprazole (5.1 Kg) was charged into a MIDAS MiKroniser-200 system-GMP Model microniser at a chamber pressure of 3.0 Kg/cm² under nitrogen pressure, and subjected to micronisation to afford 4.99 Kg of dexlansoprazole. The micronised material was charged into a clean vacuum tray dryer and dried at 32.5±2.5° C. under a reduced pressure of 650±50 mm Hg for 6-8 hours, to afford 4.90 Kg of the title compound. Water content 1.49%.

### Example 21

### Preparation of Amorphous Dexlansoprazole

(A) 2-[[(4-nitro-3-methyl-2-pyridinyl)methyl]thio]-1H-benzimidazole (10.3 g) and toluene (325 mL) were charged into a round bottom flask, fitted with a Dean-Stark apparatus, at 25-35° C., under a nitrogen atmosphere, and stirred for 5-10 minutes. The mixture was heated to 110° C. and subjected to azeotropic refluxing for 1-2 hours to remove water completely. The mixture was cooled to 65-70° C. and water (0.35 mL), (+)-diethyltartrate (12.58 mL) and titanium isopropoxide (11.71 mL) were added and stirred at 65-70° C. for 1 hour. The mixture was cooled to 15-25° C. and diisopropylethylamine (5.73 mL) was added, then the mixture was cooled to 0-5° C. Cumene hydroperoxide (10.38 mL) was added at 0-5° C. over 30-45 minutes, and the mixture was maintained at 0-5° C. for 3.5-4 hours under a nitrogen atmosphere. The reaction was quenched with 12.5% aqueous piperidine solution (100 mL) at a temperature below 5° C. and the temperature was raised to 25-35° C. The mixture was stirred at 25-35° C. for 10-15 minutes and organic and aqueous layers were separated. The organic layer was extracted with 12.5% aqueous piperidine solution (100 mL) at 25-35° C. and the mass was stirred for a period of 10-15 minutes. Organic and aqueous layers were separated and the organic layer was extracted with 12.5% aqueous ammonia solution (2×100 mL). The combined aqueous layer was washed with toluene (2×50 mL). Acetonitrile (60 mL) was added to the aqueous layer and the solution was cooled to 10-15° C. The pH of the solution was adjusted to 8.1 to 8.8 with acetic acid (56 mL). The mass was maintained at 25-35° C. for 2-3 hours. The formed solid was filtered and washed with water (100 mL) and dried at 45-50° C. to afford 6.0 g of 2-[(R)-[(4-nitro-3-methyl-2-pyridinyl)methyl]sulfinyl]-1H-benzimidazole. Chemical purity by HPLC 98.71%, nitrosulphide impurity of Formula (Ib) 0.05% nitrosulphone impurity of Formula (Ic) 1.18%, chiral purity by HPLC 94.79%.

(B) 2-[(R)-[(4-nitro-3-methyl-2-pyridinyl)methyl]sulfinyl]-1H-benzimidazole (50.0 g) and acetone (1100 mL) were charged into a round bottom flask and stirred at 25-35° C. for 5-10 minutes. The mixture was heated to 45-50° C. and maintained for 15-20 minutes to dissolve 2-[(R)-[(4-nitro-3-methyl-2-pyridinyl)methyl]sulfinyl]-1H-benzimidazole completely. The solution was cooled to 25-35° C., further cooled to −5 to 0° C., and maintained for 60-90 minutes at −5 to 0° C., then the formed solid was filtered and washed with prechilled acetone (100 mL). The filtrate was evaporated below 45° C. under reduced pressure to afford enantiomerically pure 2-[(R)-[[3-methyl-4-(2,2,2-trifluoroethoxy)-2-pyridinyl]methyl]sulfinyl]-1H-benzimidazole.

(B) Dimethylformamide (240 mL) was charged into a round bottom flask at 25-35° C. and was cooled to 15-20° C. 2,2,2-Trifluoroethanol (88.6 g) was added at 15-25° C. and stirred for 10-15 minutes. Potassium carbonate (122.1 g) was added at 15-20° C., then the mixture was heated to 50-55° C. and maintained at 50-55° C. for 30-45 minutes. The mixture was cooled to 25-35° C. and a solution of 2-[(R)-[(4-nitro-3-methyl-2-pyridinyl)methyl]sulfinyl]-1H-benzimidazole (40.0 g) in dimethylformamide (160 mL) was added and stirred for 5-10 minutes. The mixture was heated to 85-95° C. and maintained for 4-6 hours. The mixture was cooled to 25-35° C., was filtered and the filter washed with acetonitrile (160 mL). Water (800 mL) and carbon (12 g) were added to the filtrate and the mixture was heated to 60-70° C. and maintained for a period of 20-30 minutes. The mixture was filtered through a Hyflow bed at 60-70° C. and washed with water (400 mL). The filtrate was charged into a round bottom flask and cooled to 5-10° C. The pH of the solution was adjusted to 8 to 8.7 with 10% acetic acid (161 mL) and it was maintained for 30 minutes at 5-10° C., then further maintained for 2-3 hours at 5-10° C. The formed solid was filtered and washed with water (240 mL) and dried at 45-50° C. under reduced pressure, to afford 31.0 g of 2-[(R)-[[3-methyl-4-(2,2,2-trifluoroethoxy)-2-pyridinyl]methyl]sulfinyl]-1H-benzimidazole. Chiral purity by HPLC 100%, chemical purity by HPLC 99.49%, lansoprazole sulphide impurity of Formula (Id) 0.07%, lansoprazole sulphone impurity of Formula (Ie) 0.27%, RRT 1.98 impurity 0.05%.

(C) Dexlansoprazole (30.0 g) and acetone (270 mL) were charged into a round bottom flask and stirred for 10-15 minutes to dissolve dexlansoprazole completely at 25-30° C. The solution was filtered and the filter washed with acetone (30 mL). The filtrate was evaporated by spray-drying using a BÜCHI MINI Spray Dryer B-290 with BÜCHI Inert Loop B-295 spray-dryer. The obtained solid was dried at 25-30° C. for 4-6 hours under reduced pressure to afford amorphous dexlansoprazole. Yield 54-58.7%, chemical purity by HPLC 99.53%, moisture content 2.74%.

Operating parameters for spray dryer: aspirator 70%, feed rate 40% (12 mL/minute), inlet temperature 70° C., N₂ pressure: 6.5 Kg/cm².

### Example 22

### Preparation of a Solid Dispersion of Amorphous Dexlansoprazole with Cyclodextrin

Dexlansoprazole (60 g) and dichloromethane (600 mL) were charged into a round bottom flask and stirred at 27° C. for 10-15 minutes. Carbon (18 g) was added and stirred at 27° C. for 15 minutes. The solution was filtered through a Hyflow bed and washed with dichloromethane (100 mL), and the solvent from the filtrate was evaporated at 45° C. to afford 45 g of amorphous dexlansoprazole. The amorphous dexlansoprazole (45 g) and dichloromethane (300 mL) were charged into another round bottom flask and stirred at 27° C. for a period of 15 minutes. Cyclodextrin (45 g) and methanol (250 ml) were charged into another round bottom flask and stirred at 27° C. for 10-15 minutes. The above-prepared solution of dexlansoprazole in dichloromethane was added to the round bottom flask containing cyclodextrin and the mixture stirred at 27° C. for 15 minutes. The solvent from the mixture was evaporated, to afford 80 g of the title dispersion.

### Example 23

### Preparation of Amorphous Dexlansoprazole

Dexlansoprazole (25.0 g) and acetone (350 mL) were charged into a clean round bottom flask and stirred at 35° C. for 10 minutes to dissolve dexlansoprazole completely. The solution was filtered through a Hyflow bed and washed with acetone (25 mL). The filtrate was evaporated by spray-drying using a BÜCHI MINI Spray Dryer B-290 with BÜCHI Inert Loop B-295 spray-dryer, to afford 14.0 g of the title compound. Yield 56%, chemical purity by HPLC 98.58%.

Operating parameters for spray drier: aspirator 70%, feed rate 40% (12 mL/minute), inlet temperature 60° C., N₂ pressure: 5.0 Kg/cm².

### Example 24

### Preparation of Amorphous Dexlansoprazole

Acetone (10 L) and dexlansoprazole (2.5 Kg) were charged into a reactor and stirred for 15 minutes at 30±5° C. to dissolve dexlansoprazole completely. Activated carbon (0.25 Kg) was added. The mass was cooled to 7.5±2.5° C. and stirred for 15 minutes. The mass was filtered and the filter washed with acetone (2.5 L). The mass temperature was raised to 30±5° C. and it was subjected to spray drying using a BÜCHI MINI Spray Dryer B-290 with BÜCHI Inert Loop B-295 spray-dryer, and the solid was dried at 30±5° C. for 10 hours to afford 1.46 Kg the title compound.

Operating parameters for spray dryer: feed pressure 475±25 psi (Kg/cm²), nitrogen inlet temperature 40±5° C., feed rate: 3 L/hour, N₂ pressure 4.5±0.5 Kg/cm².

Product purity by HPLC: 99.4%, 2-mercaptobenzimidazole impurity of Formula (Im) 0.005%, mitrosulphoxide impurity of Formula (II) not detected, sulphone impurity of Formula (Ie) 0.10%, sulphide impurity of Formula (Id) 0.07%, RRT 1.98 impurity 0.03%, S-isomer content: 0.02%, water content by KF 0.8%.

Bulk density: before tapping: 0.47 g/mL, after tapping: 0.65 g/mL.

Particle size distribution: d(0.1) 0.896 μm, d(0.5) 5.308 μm, d(0.9) 12.938.

### Example 25

### Preparation of Amorphous Dexlansoprazole

Dexlansoprazole (17.0 g) and dichloromethane (340 mL) were combined and stirred at 27° C. for about 10 minutes to dissolve dexlansoprazole completely. Carbon (5.1 g) was added and stirred at 27° C. for 10-15 minutes. The solution was filtered through a Hyflow bed and the filter washed with dichloromethane (85 mL). The filtrate was distilled completely under reduced pressure at 45° C. to afford 13.9 g of amorphous dexlansoprazole. Chemical purity by HPLC 99.13%, chiral purity by HPLC 97.45%, specific surface area by the BET method 1.0608 m²/g.

### Example 26

### Preparation of Amorphous Dexlansoprazole

Dexlansoprazole (10.0 g) and dichloromethane (150 mL) were charged into a round bottom flask and stirred at 27° C. for 10 minutes to dissolve dexlansoprazole completely. About 100 ml of dichloromethane was distilled under reduced pressure at 39° C. to produce a concentrated solution of dexlansoprazole of about 50 mL. The solution was cooled to 27° C., added to chilled (0-10° C.) cyclohexane (50 mL) in another flask and stirred at 0-10° C. for 30-45 minutes. The formed solid was filtered and washed with chilled cyclohexane (10 mL), then was dried at 42° C. under reduced pressure to afford 7.2 g of the title compound.

### Example 27

### Stability Studies

Products from certain of the above examples and a crystalline dexlansoprazole, prepared according to Example 2 of U.S. Pat. No. 6,462,058, were tested for their storage stabilities. The samples were stored in tied clear polyethylene bags that were placed in sealed black polyethylene bags along with a silica gel desiccant pouch filled with nitrogen, and finally the black bags were placed into triple laminated foil bags along with a silica gel pouch and then sealed and stored in HDPE drum packages under the conditions noted in the following results tables. Samples were analyzed before storage, at intervals during storage, and after storage.

Samples were analyzed by HPLC for chemical purity, for:

a “sulfide” impurity, having a chemical name 2-[[{4-(2,2,2-trifluoroethoxy)-3-methylpyridine-2-yl}methyl]thio]-1H-benzimidazole and the following structure;

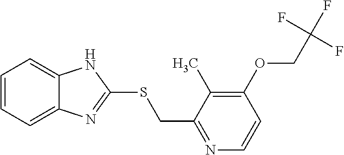

a “sulfone” impurity, having a chemical name 2-[[{4-(2,2,2-trifluoroethoxy)-3-methylpyridine-2-yl}methyl]sulfonyl]-1H-benzimidazole and the following structure;

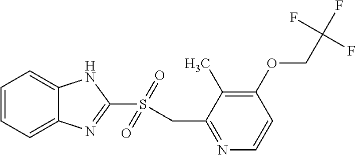

and for chiral purity. Also given in the tables is the concentration of the highest unidentified impurity, as determined by HPLC, and the PXRD analysis result. All percentages given below are expressed on a weight basis.

Initial Analysis

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.4 | 1.66 | 2.41 | 0.15 | 88.13 |
| Drug | powder | | | | | | |
| Example 8 | Light yellow | Amorphous | 97.14 | 0.61 | 1.77 | 0.24 | 99.86 |
| | powder | | | | | | |
| Example 12 | Cream color | Amorphous | 95.45 | 1.62 | 2.35 | 0.15 | 88.26 |
| | powder | | | | | | |
| Example 13 | Cream color | Amorphous | 75 | 0.58 | 24.05 | 0.2 | 98.75 |
| | powder | | | | | | |
| Example 14 | Cream color | Amorphous | 75.3 | 0.61 | 23.67 | 0.15 | 99.07 |
| | powder | | | | | | |
| Example 15 | Cream color | Amorphous | 75.23 | 0.59 | 23.83 | 0.12 | 98.96 |
| | powder | | | | | | |
| Example 16 | Light brown | Amorphous | 84.42 | 1.45 | 13.43 | 0.08 | 96.59 |
| (A) | powder | | | | | | |

After 1 Week at 2-8° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.39 | 1.68 | 2.48 | 0.14 | 88.02 |
| Drug | powder | | | | | | |
| Example 8 | Light yellow | Amorphous | 97.21 | 0.63 | 1.83 | 0.13 | 99.89 |
| | powder | | | | | | |
| Example 12 | Cream color | Amorphous | 95.35 | 1.61 | 2.35 | 0.18 | 88.4 |
| | powder | | | | | | |
| Example 13 | Cream color | Amorphous | 75.31 | 0.6 | 23.43 | 0.28 | 98.9 |
| | powder | | | | | | |
| Example 14 | Cream color | Amorphous | 74.72 | 0.65 | 23.59 | 0.34 | 99.07 |
| | powder | | | | | | |
| Example 15 | Cream color | Amorphous | 74.94 | 0.55 | 23.59 | 0.5 | 98.96 |
| | powder | | | | | | |
| Example 16 | Light brown | Amorphous | 83.76 | 1.48 | 13.78 | 0.09 | 96.57 |
| (A) | powder | | | | | | |

After 1 Week at 25-35° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.28 | 1.77 | 2.48 | 0.14 | 87.8 |
| Drug | powder | | | | | | |
| Example 8 | Light yellow | Amorphous | 97.12 | 0.68 | 1.84 | 0.14 | 99.86 |
| | powder | | | | | | |
| Example 12 | Cream color | Amorphous | 95.14 | 1.69 | 2.35 | 0.31 | 88.29 |
| | powder | | | | | | |
| Example 13 | Cream color | Amorphous | 74.9 | 0.6 | 23.73 | 0.35 | 98.93 |
| | powder | | | | | | |
| Example 14 | Cream color | Amorphous | 76.19 | 0.65 | 22.17 | 0.36 | 99.08 |
| | powder | | | | | | |
| Example 15 | Cream color | Amorphous | 75.06 | 0.56 | 23.53 | 0.23 | 98.98 |
| | powder | | | | | | |
| Example 16 | Light brown | Amorphous | 83.03 | 1.55 | 13.83 | 0.14 | 96.56 |
| (A) | powder | | | | | | |

After 2 Weeks at 2-8° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.19 | 1.62 | 2.36 | 0.13 | 88.49 |
| Drug | powder | | | | | | |
| Example 8 | Light yellow | Amorphous | 97.21 | 0.63 | 1.79 | 0.11 | 99.87 |
| | powder | | | | | | |
| Example 12 | Cream color | Amorphous | 95.27 | 1.6 | 2.28 | 0.2 | 88.48 |
| | powder | | | | | | |
| Example 13 | Cream color | Amorphous | — | — | — | — | — |
| | powder | | | | | | |
| Example 14 | Cream color | Amorphous | — | — | — | — | — |
| | powder | | | | | | |
| Example 15 | Cream color | Amorphous | — | — | — | — | — |
| | powder | | | | | | |
| Example 16 | Light brown | Amorphous | 83.6 | 1.45 | 13.63 | 0.17 | 96.86 |
| (A) | powder | | | | | | |

After 2 Weeks at 25-35° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.19 | 1.62 | 2.36 | 0.13 | 88.34 |
| Drug | powder | | | | | | |
| Example 8 | Light yellow | Amorphous | 97.23 | 1.78 | 0.63 | 0.11 | 99.86 |
| | powder | | | | | | |
| Example 12 | Cream color | Amorphous | 95.27 | 1.61 | 2.3 | 0.24 | 88.63 |
| | powder | | | | | | |
| Example 13 | Cream color | Amorphous | — | — | — | — | — |
| | powder | | | | | | |
| Example 14 | Cream color | Amorphous | — | — | — | — | — |
| | powder | | | | | | |
| Example 15 | Cream color | Amorphous | — | — | — | — | — |
| | powder | | | | | | |
| Example 16 | Light brown | Amorphous | 80.85 | 1.49 | 13.8 | 1.16 | 96.49 |
| (A) | powder | | | | | | |

After 3 Weeks at 2-8° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.65 | 1.61 | 2.31 | 0.14 | 88.34 |
| Drug | powder | | | | | | |
| Example 8 | Light yellow | Amorphous | 97.27 | 0.62 | 1.77 | 0.12 | 99.85 |
| | powder | | | | | | |
| Example 12 | Cream color | Amorphous | 95.38 | 1.59 | 2.28 | 0.14 | 88.35 |
| | powder | | | | | | |
| Example 13 | Cream color | Amorphous | — | — | — | — | — |
| | powder | | | | | | |
| Example 14 | Cream color | Amorphous | — | — | — | — | — |
| | powder | | | | | | |
| Example 15 | Cream color | Amorphous | — | — | — | — | — |
| | powder | | | | | | |
| Example 16 | Light brown | Amorphous | 83.9 | 1.5 | 13.69 | 0.07 | 96.73 |
| (A) | powder | | | | | | |

After 3 Weeks at 25-35° C.

| | | | | | | Highest | Chiral |
|---|---|---|---|---|---|---|---|
| | | | Purity | Sulfone | Sulfide | Impurity | Purity |
| Sample | Appearance | PXRD | (%) | (%) | (%) | (%) | (%) |
| Crystalline | Cream color | Crystalline | 95.4 | 1.65 | 2.39 | 0.13 | 88.27 |
| Drug | powder | | | | | | |
| Example 8 | Light yellow | Amorphous | 97 | 0.63 | 1.79 | 0.15 | 99.84 |
| | powder | | | | | | |
| Example 12 | Cream color | Amorphous | 94.99 | 1.61 | 2.3 | 0.38 | 88.21 |
| | powder | | | | | | |
| Example 13 | Cream color | Amorphous | — | — | — | — | — |
| | powder | | | | | | |
| Example 14 | Cream color | Amorphous | — | — | — | — | — |
| | powder | | | | | | |
| Example 15 | Cream color | Amorphous | — | — | — | — | — |
| | powder | | | | | | |
| Example 16 | Light brown | Amorphous | 82.07 | 1.51 | 13.69 | 0.8 | 96.67 |
| (A) | powder | | | | | | |

### Example 28

### Stability Studies

Dexlansoprazole obtained from Example 23 was tested for its storage stability. The samples were stored in a tied clear polyethylene bag, placed in a black polyethylene bag filled with N₂ along with a silica desiccant pouch, and the black bag was placed into a triple laminated bag along with silica gel pouch and then sealed and stored in HDPE drum packaging under the conditions noted in the following results tables. Chemical purity of the samples was measured by HPLC before and after storage.

The following impurities were monitored during stability testing:

2-mercaptobenzimidazole (designated “2-MB) having the following structure;

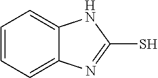

a “nitrosulphoxide” impurity (designated “NS”) having the following structure;

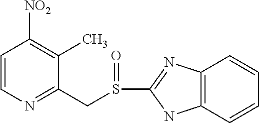

a “sulfide” impurity, having a chemical name 2-[[{4-(2,2,2-trifluoroethoxy)-3-methylpyridine-2-yl}methyl]thio]-1H-benzimidazole and the following structure;

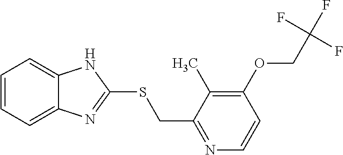

a “sulfone” impurity, having a chemical name 2-[[{4-(2,2,2-trifluoroethoxy)-3-methylpyridine-2-yl}methyl]sulfonyl]-1H-benzimidazole and the following structure;

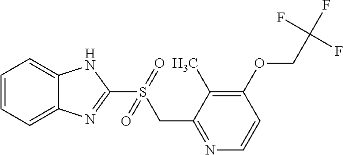

and an unidentified impurity at RRT (relative retention time) 1.98. Also given in the following tables are the concentration of the highest unidentified impurity as measured by HPLC, total impurities, and the water content. All results in the tables given below are expressed on a weight percent basis, and “ND” means not detected. The samples remained in an amorphous form during the testing, and maintained their initial cream color powder form.

At 2-8° C.

| | | | | | | RRT | Highest | Total |
|---|---|---|---|---|---|---|---|---|
| Duration | Water | 2-MB | NS | Sulfone | Sulfide | 1.98 | Impurity | impurities |
| Initial | 2.90 | 0.02 | ND | 0.59 | 0.47 | 0.03 | 0.07 | 1.42 |
| 1 month | 3.54 | 0.04 | ND | 0.57 | 0.45 | 0.06 | 0.07 | 1.54 |
| 2 months | 2.61 | 0.03 | ND | 0.61 | 0.50 | 0.06 | 0.07 | 1.76 |
| 3 months | 2.24 | 0.02 | ND | 0.59 | 0.48 | 0.06 | 0.07 | 1.63 |

At 25 ± 2° C. and 60 ± 5% Relative Humidity

| | | | | | | RRT | Highest | Total |
|---|---|---|---|---|---|---|---|---|
| Duration | Water | 2-MB | NS | Sulfone | Sulfide | 1.98 | Impurity | impurities |
| Initial | 2.90 | 0.02 | ND | 0.59 | 0.47 | 0.03 | 0.07 | 1.42 |
| 15 days | 1.68 | 0.03 | ND | 0.61 | 0.50 | 0.09 | 0.08 | 1.76 |
| 1 month | 2.89 | 0.05 | ND | 0.55 | 0.47 | 0.14 | 0.09 | 1.80 |
| 45 days | 1.29 | 0.04 | ND | 0.64 | 0.52 | 0.14 | 0.06 | 1.93 |
| 2 months | 1.54 | 0.04 | ND | 0.61 | 0.50 | 0.15 | 0.07 | 1.99 |
| 3 months | 1.50 | 0.04 | ND | 0.61 | 0.50 | 0.17 | 0.07 | 1.89 |

1. Amorphous dexlansoprazole.

2. Amorphous dexlansoprazole of claim 1, characterized by a powder X-ray diffraction pattern substantially in accordance with FIG. 1.

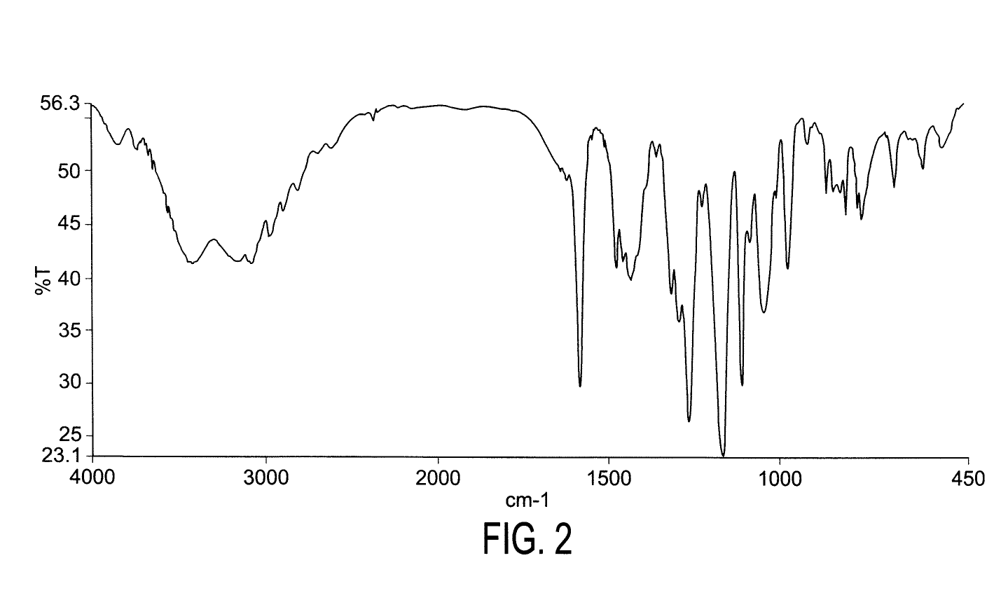

3. Amorphous dexlansoprazole of claim 1, characterized by a differential scanning calorimetry curve substantially in accordance with FIG. 2.

4. Amorphous dexlansoprazole of claim 1, characterized by an infrared absorption spectrum substantially in accordance with FIG. 3.

5. Amorphous dexlansoprazole of claim 1, characterized by a thermogravimetric analysis curve substantially in accordance with FIG. 4.

6. Amorphous dexlansoprazole of claim 1, having a water content less than about 5 percent by weight.

7. Amorphous dexlansoprazole of claim 1, substantially free of residual organic solvents.

8. A process for preparing amorphous dexlansoprazole of claim 1, comprising removing solvent from a solution containing dexlansoprazole.

9. The process of claim 8, wherein a solvent comprises any one or more of methanol, ethanol, acetone, methyl ethyl ketone, dichloromethane, chloroform, ethyl acetate, methyl acetate, acetonitrile, N,N-dimethylformamide, N,N-dimethylacetamide, dimethylsulphoxide, and water.

10. The process of claim 8, wherein solvent is removed by evaporation.

11. The process of claim 8, wherein solvent is removed by spray drying.

12. The process of claim 8, wherein solvent is removed by thin film drying.

13. The process of claim 8, wherein solvent is removed by freeze drying.

14. A process for preparing amorphous dexlansoprazole of claim 1, comprising combining a solution containing dexlansoprazole with an anti-solvent.

15. A process for preparing crystalline dexlansoprazole, comprising:
a) providing a solution comprising a salt of dexlansoprazole;
b) adding an acid to form dexlansoprazole; and
c) isolating crystalline dexlansoprazole from the solution.

16. Dexlansoprazole of claim 1 having a chemical purity greater than about 99 percent by weight, by high performance liquid chromatography.

17. Dexlansoprazole of claim 1 having an enantiomeric purity greater than about 99 percent by weight, by high performance liquid chromatography.

18. Dexlansoprazole of claim 1 having a particle size distribution with one or more of: D₁₀ being less than about 5 μm; D₅₀ being less than about 15 μm; and D₉₀ being less than about 50 μm.

19. Dexlansoprazole of claim 1 having a specific surface area greater than about 0.5 m²/g.

20. Dexlansoprazole of claim 1 having a bulk density less than about 1 g/mL.

21. A process for the preparation of amorphous dexlansoprazole of claim 1, substantially free of residual organic solvents, comprising:
a) micronizing amorphous dexlansoprazole; and
b) drying to provide amorphous dexlansoprazole substantially free of residual organic solvents.

22. A process for packaging and storing amorphous dexlansoprazole of claim 1, comprising:
a) placing dexlansoprazole into a container having an inert atmosphere, and sealing;
b) placing the sealed container and a moisture adsorbent into a second container, and sealing;
c) placing the second sealed container into a triple laminated bag and sealing; and
d) placing the triple laminated bag into a high density polyethylene container, sealing, and storing in controlled environment at about 2-8° C.

23. A process for preparing a compound of formula (I), 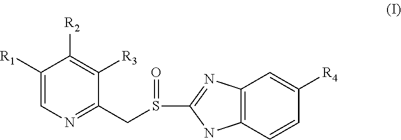
wherein each of R₁, R₂, R₃ and R₄ independently is hydrogen, C₁₋₆ alkyl or C₁₋₆ alkoxy, optionally substituted with one or more fluorine atoms, or C₁₋₆-alkoxy-C₁₋₆ alkyl groups, or a pharmaceutically acceptable salt thereof, in the form of a single enantiomer or in an enantiomerically enriched form, which includes one or more of the following steps:
a) reacting a compound of formula (II), 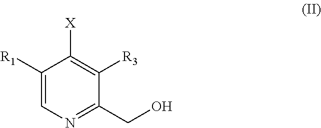
wherein R₁ and R₃ are as described previously, and X is a nitro or halo group, with:
(i) a halogenating agent; or
(ii) a compound of formula (III), 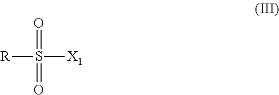
wherein X₁ is a halo group or —OSO₂R, wherein R is an alkyl group, a halogenated alkyl group, or an aryl group, optionally substituted with an alkyl group, to provide a compound of formula (IV) or a salt thereof, 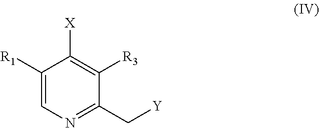
wherein Y is a halo group or —OSO₂R, wherein R is as described previously;
b) reacting a compound of formula (IV) with a 2-mercaptobenzimidazole of formula (V), 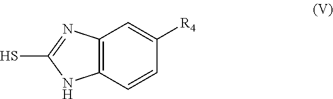
to provide a compound of formula (VI), 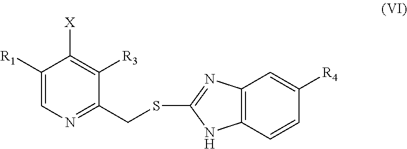
wherein R₁, R₃, R₄ and X are as described previously;
c) enantioselectively oxidizing the compound of formula (VI) with an oxidizing agent in the presence of a chiral auxiliary to provide a compound of formula (VII) in the form of a single enantiomer or in an enantiomerically enriched form, 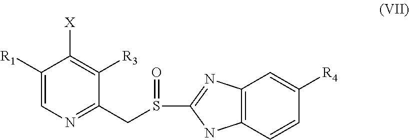
wherein R₁, R₃, R₄ and X are as described previously; and
d) reacting a compound of formula (VII), in the form of a single enantiomer or in an enantiomerically enriched form, with an alkoxide —OZ, wherein Z is a C₁₋₆ alkyl optionally substituted with one or more fluorine atoms, or C₁₋₆-alkoxy-C₁₋₆-alkyl, to provide a compound of formula (I).

24. A process for preparing a substantially pure compound of formula (VII), 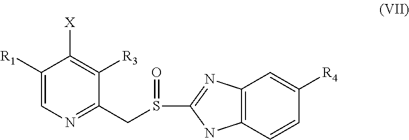
wherein each of R₁, R₃ and R₄ independently is hydrogen, C₁₋₆ alkyl or C₁₋₆ alkoxy, optionally substituted with one or more fluorine atoms, or C₁₋₆-alkoxy-C₁₋₆ alkyl groups, and X is a halo group or —OSO₂R, wherein R is an alkyl group, a halogenated alkyl group, or an aryl group, optionally substituted with an alkyl group, comprising:
a) providing a solution containing a compound of formula (VII) and a water immiscible solvent;
b) extracting the solution with an aqueous organic base;
c) separating an organic phase and adding an acid.

25. A process for optical purification of an enantiomerically enriched compound of formula (VII), 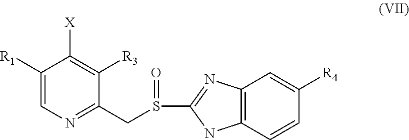
wherein each of R₁, R₃, and R₄ is independently hydrogen, C₁₋₆ alkyl or C₁₋₆ alkoxy, optionally substituted with one or more fluorine atoms, or C₁₋₆-alkoxy-C₁₋₆ alkyl groups, and X is a halo group or —OSO₂R, wherein R is an alkyl group, a halogenated alkyl group, or an aryl group, optionally substituted with an alkyl group, comprising:
a) treating an enantiomerically enriched compound of Formula (VII) with a solvent; and
b) isolating a compound of Formula (VII) with an enhanced optical purity.

26. Crystalline 2-[(R)-[(4-chloro-3-methyl-2-pyridinyl)methyl]sulfinyl]-1H-benzimidazole.

27. Crystalline 2-[(R)-[(4-chloro-3-methyl-2-pyridinyl)methyl]sulfinyl]-1H-benzimidazole of claim 26, characterized by a powder X-ray diffraction pattern having peaks located at about 6.6, 11.6, 12.2, 13.4, 14.5, 15.2, 17.2, 18.5, 19.5, 20.2, 22.1, 22.7, 23.6, 23.9, 24.8, 26.2, 26.8, 27.1, 28.4, 29.4, 30.4, 31.1, 33.7, 36.0, 37.5, 39.0, 40.2, and 41.9, ±0.2 degrees 2-theta.

28. Crystalline 2-[(R)-[(4-chloro-3-methyl-2-pyridinyl)methyl]sulfinyl]-1H-benzimidazole of claim 26, characterized by a powder X-ray diffraction pattern having peak locations substantially according to FIG. 9.

29. Crystalline 2-[(R)-[(4-chloro-3-methyl-2-pyridinyl)methyl]sulfinyl]-1H-benzimidazole of claim 26, characterized by an infrared absorption spectrum having peaks located substantially in accordance with FIG. 10.

30. Crystalline 2-[(R)-[(4-nitro-3-methyl-2-pyridinyl)methyl]sulfinyl]-1H-benzimidazole.

31. Crystalline 2-[(R)-[(4-nitro-3-methyl-2-pyridinyl)methyl]sulfinyl]-1H-benzimidazole of claim 30, characterized by a powder X-ray diffraction pattern having peaks located at about 3.2, 6.5, 9.8, 16.0, 16.5, 17.0, 17.7, 18.3, 19.3, 19.6, 20.0, 23.8, 23.9, 24.1, 24.4, 24.9, 25.4, 26.1, 26.8, 28.5, 29.0, 29.6, 30.4, 31.8, 32.6, 33.1, 33.5, 34.0, 35.0, 36.0, 36.5, 37.0, 37.6, 38.0, 39.0, 39.9, 40.7, 41.4, 42.3, and 43.5, ±0.2 degrees 2-theta.

32. Crystalline 2-[(R)-[(4-nitro-3-methyl-2-pyridinyl)methyl]sulfinyl]-1H-benzimidazole of claim 30, characterized by a powder X-ray diffraction pattern having peak locations substantially according to FIG. 11.

33. Crystalline 2-[(R)-[(4-nitro-3-methyl-2-pyridinyl)methyl]sulfinyl]-1H-benzimidazole of claim 30, characterized by an infrared absorption spectrum having peak locations substantially in accordance with FIG. 12.

34. A compound of formula (VII), 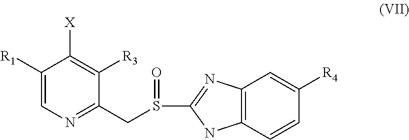
wherein each of R₁, R₃, and R₄ is independently hydrogen, C₁₋₆ alkyl or C₁₋₆ alkoxy, optionally substituted with one or more fluorine atoms, or C₁₋₆-alkoxy-C₁₋₆ alkyl groups, and X is a halo group or —OSO₂R, wherein R is an alkyl group, a halogenated alkyl group, or an aryl group, optionally substituted with an alkyl group, having a chemical purity greater than about 90 percent by weight, by high performance liquid chromatography.

35. A compound of formula (VII) of claim 34, 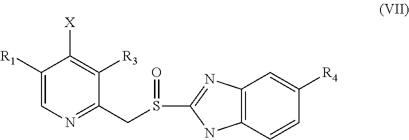
wherein each of R₁, R₃, and R₄ is independently hydrogen, C₁₋₆ alkyl or C₁₋₆ alkoxy, optionally substituted with one or more fluorine atoms, or C₁₋₆-alkoxy-C₁₋₆ alkyl groups, and X is a halo group or —OSO₂R, wherein R is an alkyl group, a halogenated alkyl group, or an aryl group, optionally substituted with an alkyl group, having an enantiomeric purity greater than about 95 percent by weight, by high performance liquid chromatography.

36. A solid dispersion comprising amorphous dexlansoprazole of claim 1 and one or more pharmaceutically acceptable carriers, with the proviso that the carrier is not a base.

37. A process for preparing a solid dispersion of claim 36, comprising providing a solution of dexlansoprazole and one or more pharmaceutically acceptable carriers, with the proviso that the carrier is not a base, and removing solvent.

38. A composition comprising dexlansoprazole substantially free of any one or more of impurities having the formulae, 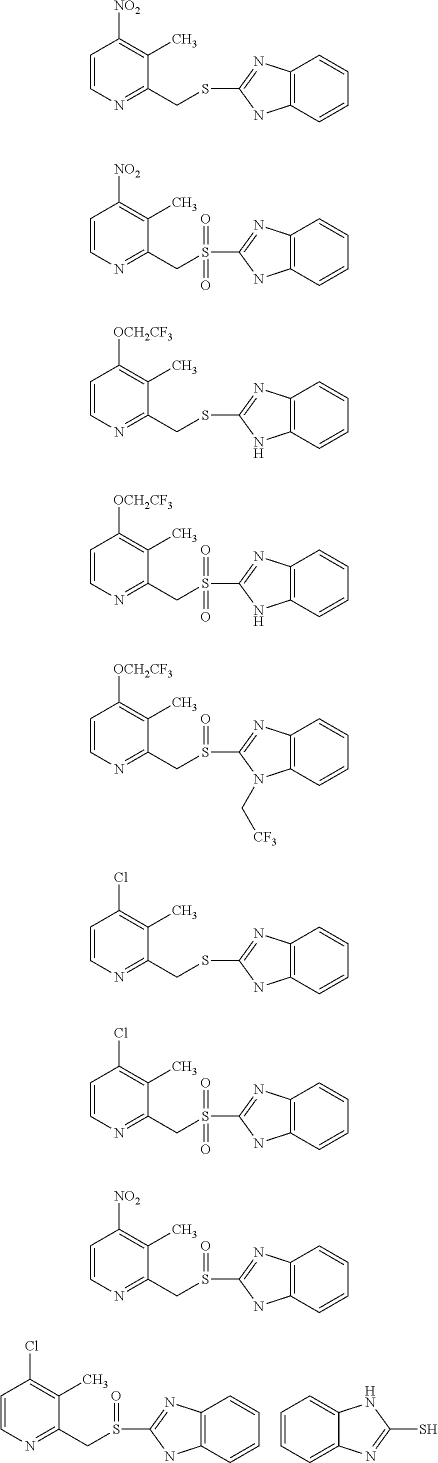 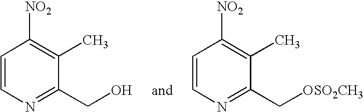
as determined by high performance liquid chromatography.

39. A pharmaceutical composition comprising dexlansoprazole of claim 18 and one or more pharmaceutically acceptable excipients.

40. A pharmaceutical composition comprising dexlansoprazole of claim 19 and one or more pharmaceutically acceptable excipients.

41. A pharmaceutical composition comprising dexlansoprazole of claim 20 and one or more pharmaceutically acceptable excipients.

42. A pharmaceutical composition comprising amorphous dexlansoprazole of claim 1 and one or more pharmaceutically acceptable excipients.

43. A pharmaceutical composition comprising a solid dispersion of claim 36 and one or more pharmaceutically acceptable excipients.
